SEQUENCE LISTING (127 sequences)

SEQ ID NO: 1 - DNA, 18 bp, artificial
  SP6 RNA promoter
atttaggtgacactatag

SEQ ID NO: 2 - DNA, 18 bp, artificial
  T3 promoter
aattaaccctcactaaag

SEQ ID NO: 3 - DNA, 29 bp, artificial
  K11 promoter
gatcaataattagggcacactatagggag

SEQ ID NO: 4 - DNA, 23 bp, artificial
  T7 promoter
gaaattaatacgactcactatag

SEQ ID NO: 5 - DNA, 18 bp, artificial
  T7 promoter
taatacgactcactatag

SEQ ID NO: 6 - DNA, 61 bp, artificial
  synthetic primer
gaaattaatacgactcactatagcgcattggcatggaagtcactttgatgcacctgtgtag

SEQ ID NO: 7 - DNA, 57 bp, artificial
  synthetic primer
cgcattggcatggaagtcactaatacgactcactatagtttgatggcacctgtgtag

SEQ ID NO: 8 - DNA, 42 bp, artificial
  synthetic primer
gaaattaatacgactcactatagttccttgtctgattagttc

SEQ ID NO: 9 - DNA, 64 bp, artificial
  synthetic primer
gaaattaatacgactcactatagtgcggccaatgtttgtaatcagccaaggaaattttggggac

SEQ ID NO: 10 - DNA, 59 bp, artificial
  synthetic primer
tgcggccaatgtttgtaatcagtaatacgactcactatagccaaggaaattttggggac

SEQ ID NO: 11 - DNA, 41 bp, artificial
  synthetic primer
gaaattaatacgactcactatagaccttcgggaacgtggtt

SEQ ID NO: 12 - DNA, 39 bp, artificial
  synthetic primer
cgcattggcatggaagtcactttgatggcacctgtgtag

SEQ ID NO: 13 - DNA, 18 bp, artificial
  synthetic primer
accttcgggaacgtggtt

SEQ ID NO: 14 - DNA, 19 bp, artificial
  synthetic primer
ttccttgtctgattagttc

SEQ ID NO: 15 - DNA, 63 bp, artificial
  synthetic primer
gaaattaatacgactcactataggcggccaatgtttgtaatcagtagacgtggtccagaacaa

SEQ ID NO: 16 - DNA, 58 bp, artificial
  synthetic primer
gcggccaatgtttgtaatcagttaatacgactcactatagagacgtggtccagaacaa

SEQ ID NO: 17 - DNA, 45 bp, artificial
  synthetic primer
gaaattaatacgactcactatagccttgtctgattagttcctggt

SEQ ID NO: 18 - DNA, 58 bp, artificial
  synthetic primer
tcagcgttcttcggaatgtcgttaatacgactcactatagctgtgtaggtcaaccacg

SEQ ID NO: 19 - DNA, 59 bp, artificial; identical to SEQ ID NO: 10
  synthetic primer

SEQ ID NO: 20 - DNA, 62 bp, artificial
  synthetic primer
gaaattaatacgactcactatagtcagcgttcttcggaatgtcgctgtgtaggtcaaccacg

SEQ ID NO: 21 - DNA, 64 bp, artificial; identical to SEQ ID NO: 9
  synthetic primer

SEQ ID NO: 22 - DNA, 41 bp, artificial
  synthetic primer
gaaattaatacgactcactatagtggcatggaagtcacacc

SEQ ID NO: 23 - DNA, 18 bp, artificial
  synthetic primer
gctgctgaggcttctaag

SEQ ID NO: 24 - DNA, 40 bp, artificial
  synthetic primer
gcggccaatgtttgtaatcagtagacgtggtccagaacaa

SEQ ID NO: 25 - DNA, 18 bp, artificial
  synthetic primer
tggcatggaagtcacacc

SEQ ID NO: 26 - DNA, 22 bp, artificial
  synthetic primer
ccttgtctgattagttcctggt

SEQ ID NO: 27 - DNA, 71 bp, artificial
  synthetic primer
gaaattaatacgactcactatagtctgacttcagtacatcaaacgaataaatacctggtgtatacgttgtc

SEQ ID NO: 28 - DNA, 66 bp, artificial
  synthetic primer
tctgacttcagtacatcaaacgaattaatacgactcactatagaaatacctggtgtatacgttgtc

SEQ ID NO: 29 - DNA, 47 bp, artificial
  synthetic primer
gaaattaatacgactcactatagtgtttcaactggttttgtgctcca

SEQ ID NO: 30 - DNA, 65 bp, artificial
  synthetic primer
gaaattaatacgactcactataggacgcgcagggaatggataattccactacttcttcagagact

SEQ ID NO: 31 - DNA, 60 bp, artificial
  synthetic primer
gacgcgcagggaatggataataatacgactcactatagttccactacttcttcagagact

SEQ ID NO: 32 - DNA, 46 bp, artificial
  synthetic primer
gaaattaatacgactcactatagtcttgcctgcgaagatctaaaac

SEQ ID NO: 33 - DNA, 19 bp, artificial
  synthetic primer
tgcaactaataaagccacg

SEQ ID NO: 34 - DNA, 48 bp, artificial
  synthetic primer
tctgacttcagtacatcaaacgaataaatacctggtgtatacgttgtc

SEQ ID NO: 35 - DNA, 23 bp, artificial
  synthetic primer
tcttgcctgcgaagatctaaaac

SEQ ID NO: 36 - DNA, 24 bp, artificial
  synthetic primer
tgtttcaactggttttgtgctcca

SEQ ID NO: 37 - DNA, 22 bp, artificial
  synthetic primer
cttctctggatttaacacactt

SEQ ID NO: 38 - DNA, 45 bp, artificial
  synthetic primer
tattggtggagctaaacttaaagccctgtacaatccctttgagtg

SEQ ID NO: 39 - DNA, 18 bp, artificial
  synthetic primer
cggtggacaaattgtcac

SEQ ID NO: 40 - DNA, 47 bp, artificial
  synthetic primer
tcagcacacaaagccaaaaatttatctgtgcaaaggaaattaaggag

SEQ ID NO: 41 - DNA, 27 bp, artificial
  synthetic primer
ttgaatttaggtgaaacatttgtcacg

SEQ ID NO: 42 - DNA, 28 bp, artificial
  synthetic primer
ttacaagcttaaagaatgtctgaacact

SEQ ID NO: 43 - DNA, 17 bp, artificial
  synthetic primer
ttgatgagctggagcca

SEQ ID NO: 44 - DNA, 18 bp, artificial
  synthetic primer
caccctcaatgcagagtc

SEQ ID NO: 45 - DNA, 41 bp, artificial
  synthetic primer
gtgtgaccctgaagactcggttttagccactgactcggatc

SEQ ID NO: 46 - DNA, 45 bp, artificial
  synthetic primer
cctccgtgatatggctcttcgtttttttcttacatggctctggtc

SEQ ID NO: 47 - DNA, 20 bp, artificial
  synthetic primer
atgtggatggctgagttgtt

SEQ ID NO: 48 - DNA, 20 bp, artificial
  synthetic primer
catgctgagtactggacctc

SEQ ID NO: 49 - DNA, 59 bp, artificial
  synthetic primer
gtgtgaccctgaagactcggtaatacgactcactatagttttagccactgactcggatc

SEQ ID NO: 50 - DNA, 63 bp, artificial
  synthetic primer
cctccgtgatatggctcttcgtaatacgactcactatagtttttttcttacatggctctggtc

SEQ ID NO: 51 - DNA, 64 bp, artificial
  synthetic primer
gaaattaatacgactcactataggtgtgaccctgaagactcggttttagccactgactcggatc

SEQ ID NO: 52 - DNA, 68 bp, artificial
  synthetic primer
gaaattaatacgactcactatagcctccgtgatatggctcttcgtttttttcttacatggctctggtc

SEQ ID NO: 53 - DNA, 43 bp, artificial
  synthetic primer
gaaattaatacgactcactatagatgtggatggctgagttgtt

SEQ ID NO: 54 - DNA, 43 bp, artificial
  synthetic primer
gaaattaatacgactcactatagcatgctgagtactggacctc

SEQ ID NO: 55 - DNA, 19 bp, artificial
  synthetic primer
aagatgagattggcatggc

SEQ ID NO: 56 - DNA, 19 bp, artificial
  synthetic primer
gcaagggacttcctgtaac

SEQ ID NO: 57 - DNA, 38 bp, artificial
  synthetic primer
ctccaaccgactgctgtctttggcttgactcaggattt

SEQ ID NO: 58 - DNA, 42 bp, artificial
  synthetic primer
cccaaagttcacaatgtggccgcatctcatatttggaatgac

SEQ ID NO: 59 - DNA, 18 bp, artificial
  synthetic primer
accttcaccgttccagtt

SEQ ID NO: 60 - DNA, 22 bp, artificial
  synthetic primer
ggactttgattgcacattgttg

SEQ ID NO: 61 - DNA, 56 bp, artificial
  synthetic primer
ctccaaccgactgctgtctaatacgactcactatagtttggcttgactcaggattt

SEQ ID NO: 62 - DNA, 60 bp, artificial
  synthetic primer
cccaaagttcacaatgtggccgtaatacgactcactatagcatctcatatttggaatgac

SEQ ID NO: 63 - DNA, 60 bp, artificial
  synthetic primer
gaattaatacgactcactatagctccaaccgactgctgtctttggcttgactcaggattt

SEQ ID NO: 64 - DNA, 65 bp, artificial
  synthetic primer
gaaattaatacgactcactatagcccaaagttcacaatgtggccgcatctcatatttggaatgac

SEQ ID NO: 65 - DNA, 41 bp, artificial
  synthetic primer
gaaattaatacgactcactatagaccttcaccgttccagtt

SEQ ID NO: 66 - DNA, 45 bp, artificial
  synthetic primer
gaaattaatacgactcactatagggactttgattgcacattgttg

SEQ ID NO: 67 - DNA, 18 bp, artificial
  synthetic primer
gtgaaggtcggagtcaac

SEQ ID NO: 68 - DNA, 18 bp, artificial
  synthetic primer
gactccacgacgtactca

SEQ ID NO: 69 - DNA, 44 bp, artificial
  synthetic primer
tgggtggaatcatattggaacaggatattgttgccatcaatgac

SEQ ID NO: 70 - DNA, 36 bp, artificial
  synthetic primer
accgtcaaggctgagaacgatctcgctcctggaaga

SEQ ID NO: 71 - DNA, 22 bp, artificial
  synthetic primer
aaccatgtagttgaggtcaatg

SEQ ID NO: 72 - DNA, 21 bp, artificial
  synthetic primer
ggaagcttgtcatcaatggaa

SEQ ID NO: 73 - DNA, 62 bp, artificial
  synthetic primer
tgggtggaatcatattggaacataatacgactcactatagggatattgttgccatcaatgac

SEQ ID NO: 74 - DNA, 54 bp, artificial
  synthetic primer
accgtcaaggctgagaacgtaatacgactcactatagatctcgctcctggaaga

SEQ ID NO: 75 - DNA, 67 bp, artificial
  synthetic primer
gaaattaatacgactcactatagtgggtggaatcatattggaacaggatattgttgccatcaatgac

SEQ ID NO: 76 - DNA, 59 bp, artificial
  synthetic primer
gaaattaatacgactcactatagaccgtcaaggctgagaacgatctcgctcctggaaga

SEQ ID NO: 77 - DNA, 45 bp, artificial
  synthetic primer
gaaattaatacgactcactatagaaccatgtagttgaggtcaatg

SEQ ID NO: 78 - DNA, 44 bp, artificial
  synthetic primer
gaaattaatacgactcactatagggaagcttgtcatcaatggaa

SEQ ID NO: 79 - DNA, 16 bp, artificial
  synthetic primer
gcgctgccaaggctgt

SEQ ID NO: 80 - DNA, 18 bp, artificial
  synthetic primer
cccaggatgcccttgagg

SEQ ID NO: 81 - DNA, 37 bp, artificial
  synthetic primer
gtggggacacggaaggccagggcaaggtcatccctga

SEQ ID NO: 82 - DNA, 41 bp, artificial
  synthetic primer
tgccaacgtgtcagtggtggactgcttcaccaccttcttga

SEQ ID NO: 83 - DNA, 21 bp, artificial
  synthetic primer
ccagtgagcttcccgttcagc

SEQ ID NO: 84 - DNA, 22 bp, artificial
  synthetic primer
tgccgtctagaaaaacctgcca

SEQ ID NO: 85 - DNA, 55 bp, artificial
  synthetic primer
gtggggacacggaaggccataatacgactcactataggggcaaggtcatccctga

SEQ ID NO: 86 - DNA, 59 bp, artificial
  synthetic primer
tgccaacgtgtcagtggtggactaatacgactcactatagtgcttcaccaccttcttga

SEQ ID NO: 87 - DNA, 60 bp, artificial
  synthetic primer
gaaattaatacgactcactataggtggggacacggaaggccagggcaaggtcatccctga

SEQ ID NO: 88 - DNA, 64 bp, artificial
  synthetic primer
gaaattaatacgactcactatagtgccaacgtgtcagtggtggactgcttcaccaccttcttga

SEQ ID NO: 89 - DNA, 44 bp, artificial
  synthetic primer
gaaattaatacgactcactatagccagtgagcttcccgttcagc

SEQ ID NO: 90 - DNA, 45 bp, artificial
  synthetic primer
gaaattaatacgactcactatagtgccgtctagaaaaacctgcca

SEQ ID NO: 91 - DNA, 43 bp, artificial
  synthetic primer
agtttctccatggagaagcgcttttctgtttgggctctctgaa

SEQ ID NO: 92 - DNA, 48 bp, artificial
  synthetic primer
tatctctacagtgaagaaacctcggttttttcttggaagctggaagac

SEQ ID NO: 93 - DNA, 18 bp, artificial
  synthetic primer
tgacgtggcaaatctagg

SEQ ID NO: 94 - DNA, 19 bp, artificial
  synthetic primer
actggggcattcttttcag

SEQ ID NO: 95 - DNA, 15 bp, artificial
  synthetic primer
gttggtggacaccgc

SEQ ID NO: 96 - DNA, 22 bp, artificial
  synthetic primer
ccatcagaaggagatgaagatt

SEQ ID NO: 97 - DNA, 61 bp, artificial
  synthetic primer
agtttctccatggagaagcgcttaatacgactcactatagtttctgtttgggctctctgaa

SEQ ID NO: 98 - DNA, 66 bp, artificial
  synthetic primer
tatctctacagtgaagaaacctcggtaatacgactcactatagttttttcttggaagctggaagac

SEQ ID NO: 99 - DNA, 66 bp, artificial
  synthetic primer
gaaattaatacgactcactatagagtttctccatggagaagcgcttttctgtttgggctctctgaa

SEQ ID NO: 100 - DNA, 71 bp, artificial
  synthetic primer
gaaattaatacgactcactatagtatctctacagtgaagaaacctcggttttttcttggaagctggaagac

SEQ ID NO: 101 - DNA, 38 bp, artificial
  synthetic primer
gaaattaatacgactcactataggttggtggacaccgc

SEQ ID NO: 102 - DNA, 45 bp, artificial
  synthetic primer
gaaattaatacgactcactatagccatcagaaggagatgaagatt

SEQ ID NO: 103 - DNA, 45 bp, artificial
  synthetic primer
tggcctcagttccgaaaaccaattttcctggataccgcagctagg

SEQ ID NO: 104 - DNA, 43 bp, artificial
  synthetic primer
ggcattcgtattgcgccgctttttggcaaatgctttcgctctg

SEQ ID NO: 105 - DNA, 18 bp, artificial
  synthetic primer
gttcaaagcaggcccgag

SEQ ID NO: 106 - DNA, 20 bp, artificial
  synthetic primer
cctccgactttcgttcttga

SEQ ID NO: 107 - DNA, 25 bp, artificial
  synthetic primer
agaaccgcggtcctattccattatt

SEQ ID NO: 108 - DNA, 19 bp, artificial
  synthetic primer
attcttggaccggcgcaag

SEQ ID NO: 109 - DNA, 63 bp, artificial
  synthetic primer
tggcctcagttccgaaaaccaattaatacgactcactatagtttcctggataccgcagctagg

SEQ ID NO: 110 - DNA, 61 bp, artificial
  synthetic primer
ggcattcgtattgcgccgctaatacgactcactatagtttttggcaaatgctttcgctctg

SEQ ID NO: 111 - DNA, 68 bp, artificial
  synthetic primer
gaaattaatacgactcactatagtggcctcagttccgaaaaccaattttcctggataccgcagctagg

SEQ ID NO: 112 - DNA, 66 bp, artificial
  synthetic primer
gaaattaatacgactcactatagggcattcgtattgcgccgctttttggcaaatgctttcgctctg

SEQ ID NO: 113 - DNA, 48 bp, artificial
  synthetic primer
gaaattaatacgactcactatagagaaccgcggtcctattccattatt

SEQ ID NO: 114 - DNA, 42 bp, artificial
  synthetic primer
gaaattaatacgactcactatagattcttggaccggcgcaag

SEQ ID NO: 115 - DNA, 32 bp, artificial
  activator sequence
gtcggttggagcgagcatcccccaaagttcac

SEQ ID NO: 116 - DNA, 53 bp, artificial
  guide RNA
uagaccaccccaaaaaugaaggggacuaaaacgtgaactttgggggatgctcg

SEQ ID NO: 117 - DNA, 53 bp, artificial
  guide RNA
uagaccaccccaaaaaugaaggggacuaaaacgaactttgggggatgctcgct

SEQ ID NO: 118 - DNA, 53 bp, artificial
  guide RNA
uagaccaccccaaaaaugaaggggacuaaaacactttgggggatgctcgctcc

SEQ ID NO: 119 - DNA, 27 bp, artificial
  activator sequence
ggccccggccccacgccaacggcaggg

SEQ ID NO: 120 - DNA, 52 bp, artificial
  guide RNA
uagaccaccccaaaaaugaaggggacuaaaacgttggcagtggggacacgga

SEQ ID NO: 121 - DNA, 52 bp, artificial
  guide RNA
uagaccaccccaaaaaugaaggggacuaaaaccacgttggcagtggggacac

SEQ ID NO: 122 - DNA, 52 bp, artificial
  guide RNA
uagaccaccccaaaaaugaaggggacuaaaactgacacgttggcagtgggga

SEQ ID NO: 123 - DNA, 27 bp, artificial
  activator sequence
ccacccaggcaaaccaggcaccgcaag

SEQ ID NO: 124 - DNA, 53 bp, artificial
  guide RNA
uagaccaccccaaaaaugaaggggacuaaaacacggtgccatggaatttgcca

SEQ ID NO: 125 - DNA, 53 bp, artificial
  guide RNA
uagaccaccccaaaaaugaaggggacuaaaacggtgccatggaatttgccatg

SEQ ID NO: 126 - DNA, 54 bp, artificial
  guide RNA
uuagaccaccccaaaaaugaaggggacuaaaactgccatggaatttgccatggg

SEQ ID NO: 127 - RNA, 54 nt, artificial
  guide RNA
agaagauagcccaagaaagagggcaauaacacauuccgaagaacgcugaagcgc

(19) United States
(12) Patent Application Publication
(10) Pub. No.: US 2023/0313282 A1
(43) Pub. Date: Oct. 5, 2023

(54) COMPOSITIONS AND METHODS OF ISOTHERMAL NUCLEIC ACID AMPLIFICATION AND DETECTION

(71) Applicant: THE REGENTS OF THE UNIVERSITY OF CALIFORNIA, Oakland, CA (US)

(72) Inventors: Patrick HSU, Berkeley, CA (US); David SAVAGE, Berkeley, CA (US); Noam PRYWES, Berkeley, CA (US); Maria LUKARSKA, Berkeley, CA (US); Shreeya AGRAWAL, Berkeley, CA (US); Sita CHANDRASEKARAN, Berkeley, CA (US); Alison FANTON, Berkeley, CA (US)

(21) Appl. No.: 18/016,776

(22) PCT Filed: Jul. 22, 2021
(86) PCT No.: PCT/US2021/042809

§ 371 Date: Jan. 18, 2023

Related U.S. Application Data
(60) Provisional application No. 63/129,910, filed on Dec. 23, 2020.
(60) Provisional application No. 63/055,088, filed on Jul. 22, 2020.

(51) Int. Cl. C12Q 1/6853

(52) U.S. Cl. CPC C12Q 1/6853

(57) ABSTRACT

Described herein are nucleic acid detection compositions and systems comprising isothermal loop mediated signal amplification (LAMP) and methods of using these LAMP-based compositions and systems.

### CROSS REFERENCE TO RELATED APPLICATIONS

This application claims the benefit of U.S. Pat. Application Serial No. 63/055,088, filed on Jul. 22, 2020. and U.S. Pat. Application Serial No. 63/129,910, filed on Dec. 23, 2020. The disclosures of the prior applications are considered part of (and are incorporated by reference in) the disclosure of this application.

### SEQUENCE LISTING

This document includes a sequence listing submitted to the United States Patent and Trademark Office via the electronic filing system as an ASCII text file. The sequence listing, which is incorporated-by-reference herein, is titled “51229-0005WO1_ST25.txt,” was created on Jul. 19, 2021, and has a size of 25 kilobytes.

### TECHNICAL FIELD

The present invention concerns methods and compositions for the targeting, amplification and/or detection of a nucleic acid, including systems comprising isothermal systems for rapid and sensitive targeting, amplification and/or detection of any target nucleic acid sequence.

### GOVERNMENT SUPPORT

Not applicable.

### BACKGROUND

Loop-mediated isothermal amplification (LAMP) is a simple and accurate isothermal nucleic acid amplification technique that has found wide spread use in laboratory and point of care settings, including for diagnostics of bacterial and viral infections such as malaria, tuberculosis, meningitis, sexually transmitted diseases (e.g., gonorrhea, chlamydia, etc.) and the like. See, e.g.. U.S. Pat. No. 6,410,278; Gadkar et al. (2018) Scientific Methods 8:5548; Notomi et al. (2006) Nucleic Acids Research 28(12):e63; Piepenburg et al. (2000) PLoS Biology 4(7):1115-1121. The LAMP technique is carried out at a single temperature (60-65° C.) and is capable of producing approximately 50 times the amount of amplified product in a short amount of time (15 minutes).

The input template for LAMP comprises DNA of a sample (or DNA that has been reverse transcribed from RNA in a sample), and is amplified into dsDNA using LAMP for detection. See. Shen (2020) J Pharm Anal 10(2):97). LAMP typically utilizes at least two pairs of primers specific to the target: (1) forward (FIP) and backward (BIP) inner primers, which hybridize to sequences in the sample; (2) forward (F3) and backward (B3) outer primers, which hybridize to sequences in the sample outside of the inner primer target sites; and optionally can use a third pair: forward (f-loop) and backward (b-loop) loop primers, which recognize sites on the loop structure and provide another layer of amplification. See, e.g., Alhassan et al. (2015) Trends Parasit doi: 10.1016/j-pt.2015.04.006). Amplification is initiated in the presence of a DNA polymerase when one of the inner primers (typically FIP) invades the double-stranded target and creates a first sequence product complementary to the target sequence that separates the double-stranded target sequence. This first sequence product is then displaced from the target sequence following extension of a product from an outer primer (F3) complementary to the target sequence. The displaced first sequence product then forms a self-hybridizing loop structure due to inclusion of reverse complementary sequence in the inner primer sequence. This cycle is repeated on the opposite end of the target sequence (with the BIP and B3 primers), which results in the formation of a dumbbell structure (also referred to as the amplicon) that includes multiple sites for initiation of synthesis. Optionally, floop and bloop primers targeting sites in the loop structure can be used to further amplify, as well as adding primers that target optional additional sites. As synthesis progresses, the products form concatemers and create more sites for initiation, thereby rapidly amplifying the target sequence into readily detectable amplicons (also referred to as “LAMPlicons”). In addition, mismatch tolerant LAMP techniques, which include adding a high-fidelity DNA polymerase to the reaction mixture that removes mismatches at the 3′ end of the LAMP primers during amplification, has been used when amplifying sequences from genetically diverse viral strains (Zhou et al. (2019) Front Micro 10, art 1056).

For the detection of RNA, RT-LAMP procedures have been developed in which an RNA polymerase promoter sequence is incorporated into the double-stranded DNA LAMP amplicon such that in the presence of the RNA polymerase, the amplicon is transcribed into an RNA product. In some embodiments, the RNA polymerase is a T7 RNA polymerase. See, e.g., Zhou et al. (2019) Front Microbiol. 10:1056, which incorporates T7 promoter sequence in F3 for detection of multiple strains Dengue virus.

LAMP techniques have also been used in combination with other nucleic acid detection systems, including PCR (see, e.g.. U.S. Pat. No. 10,100,353) as well as CRISPR-based detection systems (see, e.g., U.S. Pat. Nos. 10,253,365: 10,266,886; 10,266,887).

Rapid nucleic acid diagnostics are a critical component of a robust testing infrastructure for controlling disease transmission. For instance, to date, well over 1 million deaths from coronavirus disease 2019 (COVID-19) have been attributed to over 50 million infections of its causal virus, severe acute respiratory syndrome coronavirus 2 (SARS-CoV-2). Upon infection, individuals undergo an incubation phase when the virus is seeding and replicating in the lungs, followed by exponential viral production and reports of substantial pre-symptomatic and asymptomatic transmission (Harrison et al. (2020) Trends Immunol 41(12):1100-1115; Bai et al. (2020) JAMA: J of Am Med Assoc 323(14):1406-7). By symptom onset, viral loads in the upper respiratory tract are already declining from peak levels and accompanied by a steep drop in nucleic acid test positivity (Wölfel et al. (2020) Nature 581(7809):465-69; Zhao et al. (2020) Clinical Infectious Diseases: An Official Publication of the Infectious Diseases Society of America, March, doi .org/10.1093/cid/ciaa344).

However, some current LAMP-based detection systems may have limitations in both amplification speed and efficiencies and in regards to the conversion of the substrate into different nucleic acid types. Thus, there remains a need for LAMP-based methods and compositions that allow for the direct, sensitive, rapid, and easy-to-use amplification and detection of nucleic acids, including for the detection of DNA or RNA in a sample, including for viral pathogens such as SARS-CoV-2.

### SUMMARY

Disclosed herein are compositions and methods for targeting, isolating, amplifying and/or detecting nucleic acids (RNA and/or DNA). The compositions and methods (and systems comprising the compositions and/or method steps) involve LAMP-based techniques and systems in which one or more sequence tags are included in one or more of the LAMP primers. The systems of the invention can integrate distinct mechanisms of DNA and RNA amplification optimized for high sensitivity and rapid kinetics, linked to Cas13 detection for specificity for low-cost, widely available systems for extraction-free sample lysis and detection of nucleic acids, including for detection of SARS-Cov-2 using a system as described herein (also termed Diagnostics with Coronavirus Enzymatic Reporting (DISCoVER). The sequence tags may comprise any sequence, including, for example an RNA polymerase promoter sequence that is incorporated into the LAMP amplicon product. In the presence of the RNA polymerase, the promoter sequences in the amplicon allow for additional amplification of the target molecule and, furthermore, conversion of the target molecule into an RNA product, which in turn can be detected by RNA detection methods such as CRISPR-based RNA detection. In some cases, the RNA polymerase used is a T7 RNA polymerase, and the sequence tag incorporated into the LAMP primers comprises a T7 promoter sequence.

In any of the nucleic acid amplification/detection systems described herein the one or more sequence tags (also referred to as “oligos”) may be included in one or more of the primers of a LAMP system (e.g., FIP, BIP, F3, B3. bloop and/or floop primers). Furthermore, the sequence tag(s) may be included anywhere within the one or more primers, including at either end (5′ or 3′) of the primer(s) or inserted at any location within the primer (also referred to as “interior” or “middle” sequence tags). Any combination of the same or different sequence tags may be used in any location of one or more of the LAMP primers. In certain embodiments, at least one sequence tag is used, for example included 5′ of the FIP, interior in the FIP, 5′ of the BIP, interior in the BIP, in the floop (flanking or interior), or in the bloop (flanking or interior). In certain embodiments, the sequence tag is included 5′ to the FIP primer. In other embodiments, the sequence tag is included 5′ to the BIP primer. In other embodiments, the sequence tag is included in the interior of the FIP primer. In other embodiments, the sequence tag is included in the interior of the BIP primer. In other embodiments, the sequence is included in the floop primer. In other embodiments, the sequence is included in the bloop primer. In other embodiments, the sequence tag is included in the middle of the FIP, BIP, floop and/or bloop primers. In certain embodiments, the sequence tag is included in one or more primers as shown in FIG. 1.

In any of the systems described herein, the one or more sequence tags may comprise a sequence that acts as a binding site of a protein or a restriction enzyme site. In some embodiments, the sequence tag comprises an RNA polymerase promoter sequence. In certain embodiments, the sequence tag comprises a T7 polymerase promoter sequence that is recognized by the T7 polymerase (also referred to as RT-LAMP systems). Inclusion of the T7 sequence tag results in: (1) increased amplification efficiency (e.g.. time required to amplify the target to detectable levels of amplicon) as compared to either systems in which none of the primers include the T7 sequence or to systems in which different primer(s) include a T7 sequence: and/or (2) increased reverse transcription of the LAMP DNA amplicon into RNA (e.g., for further detection using RNA-based detection methods) as compared as compared to either systems in which none of the primers include the T7 sequence or to systems in which different primer(s) include a T7 sequence. In certain embodiments, amplification of the target sequence (creation of the LAMP amplicon) occurs more quickly as compared to systems in which the same primer(s) do not include the T7 oligo. In other embodiments, the RT-LAMP systems described herein allow for increased in vitro transcription of the RT-LAMP amplicon (as compared to systems in which the same primer(s) do not include the T7 oligo), leading to additional amplification of the target molecule into an RNA substrate.

The sequence tags (e.g., T7 polymerase promoter sequence) used in the LAMP systems described herein may be 10-50 base pairs in length (or any value therebetween). In certain embodiments, the T7 sequence tags are 20, 21, 22, 23, 24, 25. 26, 27, 28, 29, 30, 31, 32. 33, 34, 35, or more base pairs in length. In certain embodiments, the sequence tags comprise or consist of a T7 polymerase promoter sequence as shown in Table 2 below.

The LAMP (e.g., RT-LAMP) systems described herein may also be used in combination with additional nucleic acid detection techniques. In certain embodiments, the systems of the invention are used in combination with RNA detection methods, for example CRISPR-based detection methods including nuclease chain reaction (NCR) CRISPR methods. In certain embodiments, the LAMP systems described herein are used prior to the additional nucleic acid detection techniques (e.g., RT-LAMP is used prior to CRISPR-based RNA detection).

In any of LAMP systems described herein, the interaction as between one or more of the components may be limited to a certain amount of time (e.g., seconds, minutes or more). In certain embodiments, the LAMP systems provide a detectable amplicon product in 20 to 90 minutes or less (or any time therebetween, including but not limited to 20 to 30 minutes), thereby providing rapid point-of-care detection.

The target nucleic acid sequence to be amplified or detected by any of the systems described herein may be DNA and/or RNA from one or more mammals, viruses, bacteria, and/or fungi. In certain embodiments, the target sequence is in an RNA virus, for example a coronavirus, optionally a SARS-CoV-2 coronavirus. In some embodiments, the system is used to detect more than one DNA and/or RNA. In some embodiments, the system can simultaneously detect the presence of one or more nucleic acids (e.g., viral and/or other) in a multiplex manner, including systems, compositions and/or methods involving multiplexed CRISPR probes and multiplexed system reporters as well as multiplexed rLAMP primers, thereby allowing for the detection of one or more viral genes, one or more human genes (e.g., control genes) and/or one or more non-viral pathogen genes. In some embodiments, differential multiplex detection relies on different reporter systems wherein one target is detected via one reporter system and another target is detected via another reporting system wherein the detectable label generated by one reporter system is non-overlapping with the detectable label generated by the other. In some embodiments the non-overlapping detectable labels are non-overlapping fluorescent reporters. In some embodiments, differential multiplex detection relies on the use of different CRISPR-based amplification systems (e.g.. Cas13 and Cas12). In some embodiments, differential multiplex detection relies on differential rLAMP primers for amplification of two or more targets at the same time. In some embodiments, differential multiplex detection relies on a combination of two or more of differential reporter systems, differential CRISPR-based amplification systems, and/or differential rLAMP primers. In some embodiments, the system can detect a coronavirus and an influenza virus. In some embodiments, the system can detect the presense of one or more pathogens (e.g., bacterial, fungal, viral) in a sample.

In any of the systems described herein, the sample comprising the nucleic acid to be amplified and/or detected may be a biological or environmental sample. The biological sample may comprise blood, saliva, urine, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, and/or an epithelial cell sample (e.g., epithelial cell scraping), etc.) collected from the individual. In some cases, the sample is an environmental sample collected from surface swab or air samples, a waste water treatment facility or is a sample obtained from a regional sewage drainage system. The sample may be a liquid sample and may be cell-free or a liquid comprising cells. In one preferred embodiment, the methods, compositions and/or systems described herein detect a nucleic acid in an extraction-free sample, optionally an extraction-free saliva sample.

Also described are methods of amplifying one or more nucleic acids (e.g.. target sequences) in a sample, the methods comprising: (a) contacting a sample suspected of including the nucleic acid (e.g., target sequence(s)) with: (i) a LAMP-based nucleic acid detection system as described herein under conditions such that amplicons representing amplified target sequences are generated. Optionally, the methods further comprise incubating the amplicons produced with a T7 polymerase such that the amplicons are converted into an RNA substrate. In certain embodiments, the amplicons (DNA or converted to RNA) are detected, optionally by measuring a detectable signal from the detectable label (e.g.. fluorescence), thereby detecting the target sequence. In certain embodiments, the methods further comprise quantifying the levels of the detectable label.

Also described are methods of detecting a nucleic acid in a sample by (a) contacting a sample suspected of including the target sequence with an RT-LAMP system as described herein to generate an RNA substrate and (b) detecting the RNA substrate generated in step (a) using any suitable RNA detection technique (e.g.. CRISPR-based detection).

In certain embodiments, the methods of the invention include (a) contacting a sample potentially including the target sequence with: (i) any of the compositions or systems as described herein to generate amplicons representing the target sequence: and (b) measuring a detectable signal of the amplicons, thereby detecting the target sequence (DNA or RNA) in the sample. For RNA target sequences in the sample, DNA templates may be generated for LAMP-based amplification using any reverse transcriptase. In still further aspects, the methods comprise: (a) contacting a sample potentially including the target sequence with: (i) any of the compositions or systems as described herein to generate amplicons representing the target sequence and convert the amplicons to RNA; and (b) measuring a detectable signal of the amplicons, thereby detecting the target sequence in the sample. In certain embodiments, step (b) comprises detecting the amplicons (including RNA amplicons) using one or more additional nucleic acid detection systems (e.g.. CRISPR based systems).

In any of the methods described herein, the contacting step(s) may be carried out in the presence of divalent metal ions, in an acellular environment or within a cell in vitro or in vivo. The contacting step may be carried out any length of time, including seconds, minutes or hours or more (or any time therebetween), optionally seconds to 2 hours (or any time therebetween).

Accordingly, the methods and compositions of the invention comprise at least the following numbered embodiments.

### Embodiments

Described herein are systems for amplifying, isolating, targeting and/or detecting one or more target sequences (DNA or RNA) in one or more samples. In certain embodiments, provided herein is a system for amplifying at least one target nucleic acid sequence, the system comprising: (a) at least one target DNA sequence obtained from a sample, optionally wherein the target DNA sequence is transcribed from RNA obtained from the sample; at least four (optionally six or more) primers including: (i) a forward inner primer (FIP) that binds to the target DNA sequence; (ii) a backward inner primer (BIP) that binds to the target DNA sequence: (iii) a forward outer primer (F3), wherein the forward outer primer binds to the target DNA sequence 5′ to the FIP; and (iv)a backward outer primer (B3), wherein the backward outer primer binds to the target DNA sequence 3′ to the BIP; optionally further comprising: a forward loop primer (floop) and a backward loop primer (bloop), in which at least one of the primers comprises a sequence tag, optionally wherein the sequence tag is a RNA polymerase promoter sequence such as a T7 polymerase promoter, such that in the presence of a DNA polymerase, the target DNA sequence(s) is(are) amplified to generate amplicons comprising the sequence tag, optionally wherein the amplicons comprise a detectable label. The system can further comprise RNA substrate amplicons transcribed from the amplicons comprising a RNA polymerase promoter sequence. In any of the systems described herein, the sequence tag(s) may be 3′, 5′ or in the interior (anywhere not on the end) of the primer. In embodiments in which more than one sequence tag is used, the sequence tags may be used in any combinations on the same or different primers, including but not limited to one or more sequence tags on the same primer (different locations), one or more sequence tags on different primers (same or different locations as between primers), etc. In certain embodiments, the sequence tag is included in the FIP, BIP, floop or bloop primer, for example a primer as shown in Table 2.

Also described herein are methods of generating amplicons using any of the systems described herein. Methods of detecting the RNA or DNA amplicons generated using the systems described herein are also provided. In certain embodiments, the amplicons are detected directly via detectable label included in the amplicon. In other embodiments, the amplicons using a DNA or RNA detection system, optionally a CRISPR-Cas detection system, such as a nuclease chain reaction (NCR) system. In certain embodiments, the CRISPR/Cas detection system is an RNA detection system comprising a Cas protein, optionally a Cas13 protein or Csm6 protein.

In any of the methods and systems described herein, the amplicons may amplified and/or detected within 2 hours or less (e.g., within 90 minutes, 60 minutes, 45 minutes, 30 minutes, 15 minutes or less). Furthermore, in any of the methods and systems described herein, the at least one target nucleic acid sequence may be obtained from one or more mammals, viruses, bacteria, and/or fungi, including but not limited, an RNA virus, optionally a coronavirus or SARS-CoV-2 coronavirus. The sample used in any of the methods described herein may be a biological or environmental sample, for example, blood, saliva, urine, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, and/or an epithelial cell sample collected from the individual, a sample obtained from a surface, from the air, from a sewage system or from a waste water treatment facility. In any of the methods or systems described herein, the levels of the detectable label may be detected (and/or quantified).

Also provided is a kit comprising any of the systems described herein, optionally further comprising instructions for use, including instructions on performing the methods described herein.

Thus, the following numbered embodiments are included:

1. A system for amplifying at least one target nucleic acid sequence, the system comprising:
(a) at least one target DNA sequence obtained from a sample, optionally wherein the target DNA sequence is transcribed from RNA obtained from the sample;
(b) at least four primers including:
   (i) a forward inner primer (FIP) that binds to the target DNA sequence;
   (ii) a backward inner primer (BIP) that binds to the target DNA sequence;
   (iii) a forward outer primer (F3), wherein the forward outer primer binds to the target DNA sequence 5′ to the FIP;
   (iv) a backward outer primer (B3), wherein the backward outer primer binds to the target DNA sequence 3′ to the BIP; optionally further comprising
   (v) a forward loop primer (floop); and
   (vi) a backward loop primer (bloop);
wherein at least one of the primers comprises a sequence tag, optionally wherein the sequence tag is an RNA polymerase promoter sequence such as a T7 polymerase promoter, such that in the presence of a DNA polymerase, the target DNA sequence is amplified to generate amplicons comprising the sequence tag, optionally wherein the amplicons comprise a detectable label.

2. The system of claim 1, further comprising

(c) RNA substrate amplicons transcribed from the amplicons comprising the RNA polymerase promoter sequence.

3. The system of any of the preceding claims, wherein the sequence tag is 5′ of the primer.

4. The system of any of the preceding claims, wherein the sequence is interior in the primer.

5. The system of any of the preceding claims, wherein the sequence tag is included in the FIP, BIP, floop or bloop primer.

6. The system of any of the preceding claims, wherein at least one primer comprises a primer as shown in Table 2.

7. A method of amplifying one or more target sequences in a sample, the method comprising generating amplicons using the system of any of the preceding claims.

8. The method or system of any of the preceding claims, further comprising detecting the amplicons.

9. The method of system of any of the preceding claims, wherein the amplicons are detected using the detectable label.

10. The method or system of any of the preceding claims, further comprising detecting the amplicons using a DNA or RNA detection system, optionally a CRISPR-Cas detection system, such as a nuclease chain reaction (NCR) system, the CRISPR/Cas detection system comprising a Cas protein, optionally a Cas13 protein or Csm6 protein.

11. The method or system of any of the proceeding claims, further comprising multiplex detection of two or more different amplicons wherein differential detection relies on differential detectable labels, differential CRISPR-based amplifiers, and/or differential rLAMP.

12. The method or system of any of the preceding claims, wherein the amplicons are amplified and/or detected within 2 hours or less.

13. The method or system of any of the preceding claims, wherein the at least one target nucleic acid sequence is obtained from one or more mammals, viruses, bacteria, and/or fungi.

14. The method or system of any of the preceding claims, wherein the target nucleic acid sequence is from an RNA virus, optionally a coronavirus or SARS-CoV-2 coronavirus.

15. The method or system of any of the preceding claims, wherein the sample is a biological or environmental sample, for example, blood, saliva, urine, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, and/or an epithelial cell sample collected from the individual, a sample obtained from a surface, from the air, from a sewage system or from a waste water treatment facility.

16. The method or system of any of the preceding claims further comprising detecting and/or quantifying the levels of the detectable label.

17. A kit comprising a system of any the preceding claims, optionally further comprising instructions for use.

These and other aspects will be readily apparent to the skilled artisan in light of disclosure as a whole.

### BRIEF DESCRIPTION OF THE DRAWINGS

FIGS. 1A and 1B: FIG. 1A is a drawing depicting exemplary LAMP amplification using primers as described herein. The primers sets are labeled and the sequence tag (e.g., T7 promoter) is indicated. The schematic depicts six different elements (F1, F2, F3, B1, B2 and B3) within the nucleic acid sequence are replicated during LAMP. A complement (c) is made of a single strand of DNA which is then repeated over and over during the amplification process. The exemplary forward inner primer (FIP) as shown in FIG. 1A comprises F1C and F2 sequences and, as shown, may include a sequence tag interior and/or flanking (e.g., 5′) of the primer. The backward inner primer (BIP) as shown in FIG. 1A comprises B2c and Blc sequences and, as shown, may include a sequence tag interior and/or flanking (5′ end) the primer. The forward loop primer (floop) as shown in FIG. 1A comprises F2c sequences as well as F1C and F1 sequences while the backloop primer (bloop) as shown in FIG. 1A comprises B2 as well as Blc and B1 sequences. In the exemplary embodiments shown, the sequence tag (e.g.. T7 promoter sequence) is included 5′ to the floop or bloop. FIG. 1B is an illustration depicting a schematic of rLAMP mechanism for exponential DNA amplification using F3/B3 and FIP/BIP primers, resulting in higher-order inverted repeat structures. Grey arrows indicate location of T7 promoter sequence, inserted in the mBIP primer. Upon T7 transcription, the resulting RNA contains one or more copies of the Cas13 crRNA target sequence (black).

FIG. 2A through FIG. 2O are graphs showing results of time to threshold (detection) of LAMP reactions in the presence of a T7 polymerase using LAMP primers with or without a T7 sequence tag (oligo) as indicated. The grey squares show results in samples lacking the template (no template control: “NTC”) and the black circles show results using 100 copies per ul of template. Results when no T7 oligo (sequence tag) (“no T7”) was included and when the T7 oligo (sequence tag) was included 5′ in the FIP (“T7 5′FIP”); in the middle (interior) of the FIP (“T7 mFIP”); in the 5′ end of floop (“T7 floop”); in the 5′ end of BIP (“T7 5′BIP”); in the middle (interior) of the BIP (“T7 mBIP”); or in the 5′ end of floop (“T7 bloop”) are shown. FIG. 2A shows results using primers directed to the SARS-CoV-2 N Gene Set 1 (primers modified from Broughton et al. (2020) Nat Biotechnol. (38): 870-874); FIG. 2B shows results using N Gene Set 2 (primers modified from Joung et al. (2020) doi: 10.1 101/2020.05.04.20091231): and FIG. 2C shows results using Nsp3 Gene Set 1 (primers modified from Park et al. (2020) J. Mol Diagn 22(6):729-735). FIG. 2D depicts a gel showing the RNA products produced using the mBIP-T7 primer in the presense and absence of template. Also shown is the RNA product produced using the Bloop primer in the absence of template, showing that the products produced by late self-aggregation are different than those produced by the T7-LAMP method. FIGS. 2E through 2N show results from an exemplary assay as described wherein performed using saliva samples. FIG. 2E depicts an exemplary embodiment of the methods and compositions described performed on a saliva sample from a subject. Saliva samples are collected in tubes containing buffers for direct RNase inactivation and viral lysis. The rLAMP (RNA transcription following LAMP) reaction employs two mechanisms for amplification of target SARS-CoV-2 RNA. Cas13 enzymes are programmed with a guide RNA to specifically recognize the desired RNA molecules over non-specifically amplified products. Subsequent activation of Cas13 ribonuclease activity results in cleavage of reporter molecules for saturated signal within 5 minutes of CRISPR detection, enabling rapid reporting of attomolar concentrations of SARS-CoV-2. FIG. 2F depicts graphs (top panels) displaying Cas13 and Cas12 detection kinetics at varying activator concentrations. Values are mean ± SD with n = 3. The bottom panel is a graphical depiction of Cas13 and Cas12 time to half-maximum fluorescence. †, Time to half maximum fluorescence was too rapid for reliable detection. ††, Time to half maximum fluorescence could not be determined within the 120 min assay runtime. Values are mean ± SD with n = 3. FIG. 2G is a schematic of SARS-CoV-2 genome sequence. with LAMP primer set locations indicated. FIG. 2H shows representative fluorescence plots of LAMP amplification of 100 copies/uL of synthetic SARS-CoV-2 RNA or NTC. NTC, no-template control. Shaded regions denote mean ± SD with n = 3. FIG. 2I shows time-to-threshold of 9 screened LAMP primer sets, targeting synthetic SARS-CoV-2 RNA or NTC. Replicates that did not amplify are represented at 0 minutes. Error bars represent SD of amplified replicates. FIG. 2J depicts a limit of detection assay of LAMP using N Set 1 primer set. Replicates that did not amplify are represented at 0 minutes. Error bars represent SD of amplified technical replicates. FIG. 2K is a schematic of the location of different T7 promoter locations on the rLAMP dumbbell structure and loop primers. FIG. 2L is a graph depicting rLAMP time to threshold of 6 distinct T7 promoter insertions. Replicates that did not amplify are represented at 0 minutes. Error bars represent SD of amplified technical replicates. FIG. 2M, left, is a schematic of primary mBIP rLAMP products and sizes upon Afel digestion (left). Middle panel shows a virtual gel depicting expected AfeI restriction digest bands (147 nt, 128 nt) and resulting transcription bands of mBIP rLAMP products. Only the 147 nt product is expected to contain the T7 promoter, and thus produce an ~85nt RNA product upon transcription. Right panel shows a restriction-digestion of each rLAMP product with each primer set containing the different T7 insertion positions. FIG. 2N shows denaturing PAGE gels of mBIP rLAMP products to verify successful T7-mediated transcription. AfeI cleaves in the crRNA target region of templated products, which is expected to result in a single major transcribed species. FIG. 2O is a graph showing the kinetics of T7 transcription and Cas13 detection on mBIP rLAMP products. RNP, ribonucleoprotein. Values are mean ± SD with n = 3.

FIG. 3A through FIG. 3F are graphs showing Cas13 detection of in vitro transcribed RT-LAMP/T7 amplicons via fluorescence. Results are shown for the reaction of the system described herein as well as the following control traces. Error bars represent SD of amplified technical replicates. The controls indicate that experiments that lack the activator target for the Cas13 RNP (“no activator control”), that lack the Cas13 detector (“no RNP”), that lack the T7 promoter sequences in the primer (“LAMPlicon- no T7 promoter) and that lack the target sequence (“NTC”) do not yield threshold results in the same time frame as those that include all the components of the system. FIG. 3A shows results where the T7 sequence tag was included 5′ in the FIP (“5′FIP”). FIG. 3B shows results where the T7 sequence tag was included in the middle (interior) of the FIP (“mFIP”). FIG. 3C shows results where the T7 sequence tag was included 5′ in the BIP (“5′BIP”). FIG. 3D shows results where the T7 sequence tag was included in the middle (interior) of the BIP (“mBIP”). FIG. 3E shows results where the T7 sequence tag was included in the floop. FIG. 3F shows results where the T7 oligo was included in the bloop.

FIG. 4A through FIG. 4F display conditions and results following extraction-free saliva detection. FIG. 4A displays results from direct saliva lysis conditions tested for compatibility with the DISCoVER workflow. Replicates that did not amplify are represented at 0 minutes. Error bars represent SD of amplified technical replicates. IA: inactivation reagent: QE: QuickExtract. FIG. 4B is a schematic of contrived saliva sample generation, quantification via qRT-PCR, and detection via DISCoVER to determine analytical sensitivity. FIG. 4C is a graphical display of the fold-change in DISCoVER signal relative to NTC on SARS-CoV-2 positive saliva samples at 5 minutes of Cas13 detection. FIG. 4D is a similar display of the fold-change in DISCoVER signal relative to NTC on 30 negative saliva samples, at 5 minutes of Cas13 detection. FIG. 4E is a schematic of (LAMP multiplexing with SARS-CoV-2 (N Gene) and human internal control (RNase P) primer sets. FIG. 4F is a graph showing DISCoVER signal of SARS-CoV-2 positive saliva samples after multiplexed rLAMP. Values are mean ± SD with n = 3.

FIG. 5A through FIG. 5F are graphs showing duplicate experiments of Cas13 detection of in vitro transcribed RT-LAMP/T7 amplicons via fluorescence as described in FIGS. 3A through 3F. FIG. 5A shows results where the T7 sequence tag was included 5′ in the FIP (“5′FIP”). FIG. 5B shows results where the T7 sequence tag was included in the middle (interior) of the FIP (“mFIP”). FIG. 5C shows results where the T7 sequence tag was included 5′ in the BIP (“5′BIP”). FIG. 5D shows results where the T7 sequence tag was included in the middle (interior) of the BIP (“mBIP”). FIG. 5E shows results where the T7 sequence tag was included in the floop. FIG. 5F shows results where the T7 oligo was included in the bloop.

FIG. 6A through FIG. 6B are graphs showing multiplex LAMP amplification followed by LbuCas13a detection. FIG. 6A shows results of N gene LAMPlicon detection and demonstrated that Cas13 complexed with N Gene LAMPlicon targeting guide was able to detect the amplified material at the previously established LOD of 40 cp/uL (filled triangle, inverted triangle, diamond). Positive controls (filled circle, open square) and negative controls (filled square, open circle) are also depicted.FIG. 6B shows results of RNase P LAMPlicon detection, demonstrating that Cas13 complexed with the RNase P targeting guide (filled square, triangel, inverted triangle) was able to detect human RNase P LAMPlicon. Positive controls (diamond and open square) and negative controls (filled circle, open circle) are also plotted in the figure.

FIG. 7A through FIG. 7C are graphs showing LbuCas13 + LbaCas13a targeting N Gene and RNaseP LAMPlicons. FIG. 7A shows results of detection of a SARS-CoV-2 N-gene LAMPlicon using a 5-U FAM reporter. Only detection of the FAM signal is seen, indicating that LbuCas13a is specifically detecting the N Gene LAMPlicon without off-target and background HEX reporter cleavage. FIG. 7B shows results of detection of the human RNase P LAMPlicon using a 5-A HEX reporter. Only detection of the HEX signal is seen, indicating that the LbaCas13a is specifically detecting the RNase P LAMPlicon. FIG. 7C shows multiplexed detection results of both the N-gene and RNase pLAMPlicons using a 5-U FAM reporter and a 5-A HEX reporter.

### DETAILED DESCRIPTION

Described herein are compositions, systems and methods for the efficient and rapid: amplification of target sequences (LAMP amplicons); conversion of these LAMP amplicons into RNA substrates: and detection of LAMP amplicons and/or the converted RNA substrates. Thus, the compositions, systems and methods described herein provide for the efficient, sensitive and rapid detection of any target DNA or RNA, including for detection of transcriptional states, cancers, or pathogens such as bacteria or viruses, including coronaviruses such as SARS-CoV-2 (associated with COVID-19 disease).

The LAMP-based compositions described herein allow for the rapid (30 minutes or less) and sensitive (sensitivity of 20 aM or less) amplification of small quantities of nucleic acids. The amplified DNA or RNA product (amplicons) can be directly detected or, alternatively, can be detected using one or more additional nucleic acid detection systems, for example CRISPR-Cas detection, molecule beacons, FRET probes, split-fluorescent protein probes and/or RNA aptamers. Thus, the LAMP-based systems and methods described herein provide rapid, efficient and sensitive diagnostics suitable for both traditional and point-of-care applications.

In the case of SARS-CoV-2 infection, the challenges of a high asymptomatic infection rate (Lavezzo et al. (2020) Nature 584(7821):425-29), insufficient testing, and the narrow time window when molecular tests have high sensitivity have been exacerbated by the long sample-to-answer time of the centralized diagnostic laboratory model. Laboratory developed tests such as quantitative PCR (qPCR) are conducted in facilities that require laborintensive personnel and equipment infrastructure for sample accessioning, nucleic acid extraction, thermocycling, and data analysis.

Coupled with direct lysis and saliva sampling. CRISPR-based detection and isothermal amplification have significant potential for point-of-care diagnostics. CRISPR enzyme-based nucleic acid detection relies on the guide RNA-dependent activation of Cas13 or Cas12 nucleases to induce non-specific ssRNA or ssDNA nuclease activity, respectively, in order tocleave and release a caged reporter molecule (East-Seletsky et al. (2016) Nature 538(7624):270-273; Gootenberg et al. (2017) Science 356(6336):438-42; Chen et al. (2018) Science 360(6387):436-39). The released reporter can be detected with fluorescent or lateral flow assays to read out the test result. CRISPR-based detection is highly specific, but Cas13 nucleases alone can take up to two hours to reach attomolar sensitivity for diagnostic applications (Fozouni et al. (2020) MedRxiv doi: doi.org/10.1101/2020.09.28.20201947). In contrast, loop-mediated isothermal amplification (LAMP) performs highly sensitive nucleic acid amplification in under 20 minutes with attomolar limits of detection (LODs) (Nagamine et al. (2002) Mol Cell Probes 16(3):223-29). However, despite the sensitivity and speed of LAMP, such isothermal methods are often prone to non-specific amplification (Hardinge and Murray (2019) Sci Report 9(1):7400).

Fast, frequent and point-of-care testing, such as upon entry into the workplace or classroom, is postulated to be an effective way to break the chain of transmission (Larremore et al. (2020) medRxiv: The Preprint Server for Health Sciences, June. doi.org/10.1101/2020.06.22.20136309).

There is also tremendous potential for community surveillance testing to augment clinical workflows, where positive cases are confirmed by referral to a more constrained supply of clinical-grade tests. Alternative sampling methods and test technologies can also help diversify the diagnostic supply chain, as the standard pipeline for clinical testing has proven vulnerable to reagent constraints such as RNA extraction kit or swab shortages (Vandenberg et al. (2020) Nat Rev Microbiol doi:10.1038/s41579-020-00461-z). Thus, the compositions and methods described herein provide diagnostic tests that may be used to break the chain of transmission of an infectious agent such as the SARS-CoV-2 or influenza viruses by providing a method for community surveillance.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art.

### Definitions

“Oligonucleotide,” “polynucleotide” and “nucleic acid” are used interchangeably herein. These terms may refer to a polymeric form of nucleic acids of any length, strandedness (double or single), and either ribonucleotides (RNA) or deoxyribonucleotides (DNA), and hybrid molecules (comprising DNA and RNA). The disclosed nucleic acids may also include naturally occurring and synthetic or non-natural nucleobases. Natural nucleobases include adenine (A), thymine (T), cytosine (C), guanine (G), and uracil (U).

“Complementarity” refers to a first nucleic acid having a first sequence that allows it to “base pair,” “bind,” “anneal”, or “hybridize,” to a second nucleic acid. Binding may be affected by the amount of complementarity and certain external conditions such as ionic strength of the environment, temperature, etc. Base-pairing rules are well known in the art (A pairs with T in DNA, and with U in RNA: and G pairs with C). In some cases, RNA may include pairings where G may pair with U. Complementarity does not in all cases, indicate complete or 100% complementarity. For example, complementarity may be less than 100% and more than about 60%.

“Protein,” “peptide,” “polypeptide” are used interchangeably. The terms refer to a polymeric form of amino acids of any length, which may include natural and non-natural residues. The residues may also be modified prior to, or after incorporation into the polypeptide. In some embodiments, the polypeptides may be branched as well as linear.

“Programmed,” in reference to a Cas protein, refers to a Cas protein that includes a guide RNA that contains a sequence complementary to a target sequence. Typically, a programmed Cas protein includes an engineered guide RNA.

“Cas protein” is a CRISPR associated protein. The presently disclosed Cas proteins possess a nuclease activity that may be activated upon binding of a target sequence to a guide RNA bound by the Cas protein. As disclosed in more detail below, the guide RNA may, with other sequences, comprise a crRNA, which may, in some embodiments, be processed from a pre-crRNA sequence. In an embodiment, the guide RNA sequence may include natural or synthetic nucleic acids, for example modified nucleic acids such as, without limitation, locked nucleic acids (LNA), 2′-o-methylated bases, or even ssDNA (single stranded DNA). Cas proteins may be from the Cas12 or Cas13 group, which may be derived from various sources known to those of skill in the art.

The Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The tenn “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

“Coding sequences” are DNA sequences that encode polypeptide sequences or RNA sequences, for example guide RNAs. Coding sequences that encode polypeptides are first transcribed into RNA, which, in-turn, may encode the amino acid sequence of the polypeptide. Some RNA sequences, such as guide RNAs may not encode amino acid sequences.

“Native,” “naturally-occurring,” “unmodified” or “wild-type” describe, among other things, proteins, amino acids, cells, nucleobases, nucleic acids, polynucleotides, and organisms as found in nature. For example, a nucleic acid sequence that is identical to that found in nature, and that has not been modified by man is a native sequence.

By “hybridizable” or “complementary” or “substantially complementary” it is meant that a nucleic acid (e.g., RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule: of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more. 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al. (1990) J. Mol. Biol. 215:403-410; Zhang and Madden (1997) Genome Res. 7:649-656) or by using the Gap program (Wisconsin Sequence Analysis Package, Version 8 for Unix, Genetics Computer Group. University Research Park, Madison Wis.), e.g., using default settings, which uses the algorithm of Smith and Waterman (Adv. Appl. Math., 1981, 2, 482-489).

“Binding” as used herein refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g.. when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10-6 M, less than 10-7 M, less than 10-8 M, less than 10-9 M, less than 10-10 M, less than 10-11 M, less than 10-12 M, less than 10-13 M, less than 10-14 M. or less than 10-15 M. “Affinity” refers to the strength of binding, increased binding affinity being correlated with a lower Kd.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The terms “DNA regulatory sequences,” “control elements” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner For instance, a promoter is operably linked to a coding sequence if the promoter affects its transcription or expression.

The terms “recombinant expression vector” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Any given component, or combination of components can be unlabeled, or can be detectably labeled with a label moiety. In some cases, when two or more components are labeled, they can be labeled with label moieties that are distinguishable from one another.

“Label” or “labelling” refers to a component with a molecule that renders the component identifiable by one or more techniques. Non-limiting examples of labels include streptavidin and fluorescent molecules. The term “fluorescer” refers to a substance or a portion thereof which is capable of exhibiting fluorescence in the detectable range. The labels may be detected by a binding interaction with a label (e.g., biotin binding streptavidin) or through detection of a fluorescent signal using a fluorimeter. Other detectable labels include enzymatic labels such as luciferase, peroxidase or alkaline phosphatase. A “reporter gene” or “reporter sequence” refers to any sequence that produces a protein product that is easily measured, preferably although not necessarily in a routine assay. Suitable reporter genes include, but are not limited to, sequences encoding colored or fluorescent or luminescent proteins (e.g., green fluorescent protein, enhanced green fluorescent protein, red fluorescent protein). In some embodiments, enzymatic labels are inactivated by way of being split into two or more pieces that are linked by a nucleic acid linker that is targetable by an enzyme activity (e.g.. trans cleavage by a Cas protein). Upon cleavage of the linker, the pieces of the enzymatic reporter would be able to assemble into an active enzyme that could act on a substrate to generate a detectable signal.

The term “sample” is used herein to mean any sample that includes RNA and/or DNA (e.g., in order to determine whether a target sequence is present among a population of polynucleotide sequences). The sample can be derived from any source, e.g., the sample can be a synthetic combination of purified RNAs/DNAs; the sample can be a cell lysate, an RNA/DNA-enriched cell lysate, or RNA/DNAs isolated and/or purified from a cell lysate. The sample may be an environmental sample, an agricultural sample or a food sample. The sample can be from a patient (e.g., for the purpose of diagnosis). The sample may be selected or derived from one or more of blood, sweat, plasma, serum, sputum, saliva, mucus, cells, excrement, urine, cerebrospinal fluid (CSF), breast milk, semen, vaginal fluid, tissue, etc. The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example. Shah et al. (2016) Development 143:2862-2867 doi: 10.1242/dev. 138560.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an” and “the” include plural referents unless the context clearly dictates otherwise. Thus, for example, reference to a “Cas protein” includes a plurality of Cas proteins (including the same or different Cas effector proteins) and reference to “the guide RNA” includes reference to one or more guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. The compositions, methods, and systems for detecting the presence or absence of specific target nucleic acid sequence (e.g., RNA or DNA) in a sample allow for cost-effectively diagnosing a patient or sample having a viral, bacterial, parasitic, or fungal infection, or a condition, disease, or disorder by identification by the presence of one or more specific nucleic acid sequences. The compositions, methods and systems of the invention are also useful in genetic screening, cancer screening, mutational analysis, microRNA analysis, mRNA analysis, single nucleotide polymorphism analysis, etc.

### Compositions and Systems

Provided are compositions, systems and methods for amplifying and/or detecting a target RNA or DNA sequence (double stranded or single stranded) in a sample. In particular, described herein are LAMP-based systems that include one or more sequence tags in (flanking or within) one or more of the primers (FIP, BIP, F3, B3, bloop and/or floop) of the LAMP-based system. The systems and methods can comprise any number and type of sequence tags, for example an RNA polymerase promoter sequence, in one or more of the LAMP primers. In some embodiments, the RNA polymerase is the T7 polymerase. The LAMP-based systems described herein can also be used in conjunction with additional detection methods, including RT-LAMP systems that can be used to generate RNA sequences for detection with CRISPR (Cas) proteins.

Thus, the disclosed systems provide for inexpensive, sensitive and rapid detection of nucleic acid target sequences from a variety of sources including mammals, viruses, bacteria, fungi, etc. The samples may be biological samples from a human or non-human patient, or an environmental sample from water, food, etc.

### Lamp/RT-Lamp

The LAMP compositions and systems described herein comprise at least 3 primer pairs, namely forward and backward inner primers (FIP, BIP), forward and backward outer primers (F3, B3) and forward and backward loop primers (floop and bloop). Optionally, additional primers are also included. Furthermore, one or more of the primers may be designed so include additional primer or initiation sites therein, such that in the presence of a DNA polymerase, DNA amplification occurs.

Any sequence tag (oligo) of any length may be included in one or more of the primers of a LAMP system (e.g.. FIP, BIP, F3, B3, bloop and/or floop primers). Furthermore, the sequence tag(s) may be included anywhere within the one or more primers, including at either end (5′ or 3′) of the primer(s) or inserted at any location within the primer (also referred to as “interior” or “middle” sequence tags). Any combination of the same or different sequence tags may be used in any location of one or more of the LAMP primers. For example, at least one sequence tag may be included 5′ of the FIP, interior in the FIP, 5′ of the BIP, interior in the BIP, in the floop (flanking or interior), or in the bloop (flanking or interior). In certain systems (RT-LAMP systems) as described, one sequence tag is included in the system, including, by way of example 5′ to the FIP primer; 5′ to the BIP primer; in the interior of the FIP primer; in the interior of the BIP primer; in the floop primer; or in the bloop primer.

In any of the systems described herein, the one or more sequence tag may comprise a sequence that acts as a binding site of a protein. RT-LAMP systems comprise (typically incorporated into one or more primers of the LAMP system) one or more RNA polymerase promoter sequences that are recognized by the RNA polymerase. In some embodiments, the RNA polymerase used is the SP6 RNA polymerase, and the sequence tag comprises a SP6 RNA promoter (e.g., 5′ ATTTAGGTGACACTATAG 3′ (SEQ ID NO:1)). In some embodiments, the RNA polymerase used is a T3 RNA polymerase, and the sequence tag comprises a T3 promoter (e.g.. 5′ AATTAACCCTCACTAAAG 3′, (SEQ ID NO:2)). In some embodiments, the RNA polymerase used is a K11 RNA polymerase, and the sequence tag comprises a K11 promoter (e.g.. 5′ GATCAATAATTAGGGCACACTATAGGGAG-3′ (SEQ ID NO:3). In some embodiments, the RNA polymerase used is a T7 RNA polymerase, and the sequence tag comprises a T7 promoter (e.g., 5′ GAAATTAATACGACTCACTATAG-3′ (SEQ ID NO:4) or 5′ TAATACGACTCACTATAG-3′ (SEQ ID NO:5)). (See Jorgensen et al. (1991) J Biol Chem 266 (1):645-651; Goo Han (2002) J Biochem Mol Biol 35(6):637-641). Inclusion of the T7 sequence tag in one or more primers of the RT-LAMP system results in: (1) increased amplification efficiency of the amplicon (e.g., time required to amplify the target to detectable levels of amplicon) as compared to either systems in which none of the primers include the T7 sequence or to systems in which different primer(s) include a T7 sequence: and/or (2) increased reverse transcription of the LAMP DNA amplicon into RNA (e.g., for further detection using RNA-based detection methods) as compared as compared to either systems in which none of the primers include the T7 sequence or to systems in which different primer(s) include a T7 sequence.

The sequence tags (e.g., T7 polymerase promoter sequence) used in the LAMP systems described herein may be 10-50 base pairs in length (or any value therebetween). In certain embodiments, the T7 sequence tags are 20, 21, 22, 23, 24, 25. 26, 27. 28, 29, 30, 31, 32. 33, 34. 35, or more base pairs in length. In certain embodiments, the sequence tags comprise or consist of the T7 polymerase promoter sequence as shown in Table 2 below.

In some embodiments, the compositions described herein are used with alternative amplification systems. For example, Rrecombinase polymerase amplification (RPA) is the isothermal amplification of specific DNA fragments achieved by the binding of opposing oligonucleotide primers to template or target DNA and their extension by a DNA polymerase. Global melting of the amplification template is not required for the primers to be directed to their complementary target sequences. Instead, RPA employs recombinase-primer complexes to scan the doublestranded DNA and facilitate strand exchange at cognate sites. The resulting structures are stabilized by single-stranded DNA binding proteins interacting with the displaced template strand, thus preventing the ejection of the primer by branch migration. Recombinase disassembly leaves the 3′ end of the oligonucleotide accessible to a strand displacing DNA polymerase, in this case the large fragment of Bacillus subtilis Pol I (Bsu), and primer extension ensues. Exponential amplification is accomplished by the cyclic repetition of this process. Thus, in some embodiments, amplification of the target nucleic acid may be performed using LAMP, RPA or other isothermal amplification techniques including strand displacement amplification (SDA, Walker et al. (1992) PNAS USA 89:392-396), helicase-dependent amplification (Vincent et al. (2004) EMBO Rep 5:795-800) and multiple strand amplification (MDA, Dean et al. (2002) PNAS USA 99(8):5261-6).

### Additional Detection Methods

The LAMP-based systems described herein can be used in combination with one or more additional nucleic acid detection systems, for example, PCR, or systems such as CRISPR detection systems (RNA or DNA). In some embodiments, the LAMP-based system described is used with SHERLOCK (Kellner et al. (2019) Nat Protocol 14(10): 2986-3012), with DETECTR (Chen et al. (2018) Science 360 (6387):436-439) or with HOLMESv2 (Li et al. (2019) ACS Synth Biol 8(10):2228-2237). See, also, medrxiv.org/content/10.1101/2020.12.14.20247874v1. In some embodiments, sequence specific aptamer-based fluorescent assays are used for detection (Filonov et al. (2014) J Am Chem Soc 136(46):16299-16308; Park (2018) Biosens Bioelectron 102:179-188 (review)). In some embodiments, quantum dot-based detection systems are used (Freeman et al. (2013) ACS Appl Mater Interfaces 5(8):2815-34). In some embodiments, amplification is detected using an intercalating agent (e.g.. ethidium bromide or SYBR™ Safe (Invitrogen). In some embodiments, the amplification is detected and quantitated using Taqman (ThermoFisher). LAMP-based systems may be used before, after or between the one or more additional detection systems.

In one aspect, the LAMP systems described herein are used prior to detection using a DNA or RNA detector, such as a CRISPR (Cas) detection system. In certain embodiments, the LAMP system is an RT-LAMP system and the detector is an RNA CRISPR (Cas detection systems).

Any Cas protein(s) can be used in the CRISPR detection system, including but not limited to Cas proteins from any type of CRISPR/Cas system (e.g., Type II, Type III, Type V, Type VI), Csm6 proteins, Csx1 proteins and the like.

The Cas proteins may be derived from any suitable source, including archaea and bacteria. In some embodiments, a native Cas protein may be derived from Paludibacter, Carnobacterium, Listeria, Herbinix, Rhodobacter. Leptotrichia, Lachnospiraceae, Eubacterium, or Clostridium. In some embodiments, the native Cas protein may be derived from Paludibacter propionicigenes. Carnobacterium gallinarum, Listeria seeligeri. Listeria newyorkensis. Herbinix hemicellulosilytica, Rhodobacter capsulatus, Leptotrichia wadei, Leptotrichia buccalis, Leptotrichia shahii, Lachnospiraceae bacterium NK4A 179, Lachnospiraceae bacterium MA2020, Eubacterium rectale, Lachnospiraceae bacterium NK4A 144, and Clostridium aminophilum.

The Cas protein(s) as described herein may be homologous to a native Cas protein. In some embodiments, the disclosed Cas protein is greater than 75%, 80%, 85%, 90%, 95%. 97%, 98%, or 99%, and less than about 100%, 99%, 98%, 97%, 95%, 90%, 85%, 80%, or 75% identical to a native Cas protein sequence. The disclosed Cas protein may have one or more HEPN domains, and may be able, after activation, to cleave single stranded RNA, including precursor guide RNA and indicator RNA.

Activation of a Cas protein may include contacting one or more target sequences with a guide RNA sequence associated with the Cas protein. In some embodiments, the guide RNA of the Cas protein may help to activate the Cas protein’s RNase activity by hybridizing to a complementary target RNA sequence.

The disclosed Cas proteins may be any Cas protein, including but not limited to Type V (e.g., Cas12 and/or Cas14), Type VI (e.g., Cas13), and/or Type III (e.g., Csm6) proteins.

In certain embodiments, the additional nucleic acid detection systems include one or more Cas13 protein with 4 currently characterized subtypes (Cas13a-d) that each exhibit significant sequence divergence apart from two consensus HEPN (Higher eukaryotes and prokaryotes nucleotide-binding domain) RNase motifs, R-X4-6-H. To defend against viral infection, Cas13 enzymes process precrRNA into mature crRNA guides in a HEPN-independent manner, followed by HEPN-dependent cleavage of a complementary “activator” target RNA in cis. Upon target-dependent activation, Cas13 is also able to cleave bystander RNAs in trans, reflecting a general RNase activity capable of both cis- and trans-cleavage. (See, e.g.. U.S. Pat. Publication No. 2020/0032324 and International Patent Publication No. WO 2017/218573, Konnermann et al. (2018) Cell Apr 19: 173(3):665-676: Zhang et al. (2018) Cell 175(1):212-223). The signature protein of Type VI-A CRISPR-Cas systems, Cas 13a (formerly C2c2), is a dual nuclease responsible for both crRNA maturation and RNA-activated ssRNA cleavage (East-Seletsky et al. (2016) Nature 538(7624):270-273). Casl3a binds to precursor crRNA (pre-crRNA) transcripts and cleaves them within the repeat region to produce mature crRNAs. When the pre-crRNA is processed to the individual mature crRNAs, an 8-nucleotide piece of the repeat region that separates each of the spacer regions in a CRISPR array remains attached to the mature crRNA and is termed the “tag”. Binding to a ssRNA activator (target) sequence with complementarity to the crRNA activates Cas13a for trans-ssRNA cleavage, potentially triggering cell death or dormancy of the host organism. However, if the target or activator RNA comprises a sequence that is complementary to the tag sequence (known as the “anti-tag”) the complex is inhibited from being activated. This is thought to be a mechanism involved in preventing autoimmunity (Meeske & Marriffini (2018) Mol Cell 71:791). The Cas13a’s trans-ssRNA activity can be exploited for use in releasing cage structures on RNAs: an activity that can be tunes by use of cage sequences that correspond to the preferences for the different Cas13a homologs.

In some embodiments, the Cas13 protein is a Cas13a polypeptide comprising an amino acid sequence having at least 75%, at least 80%, at least 85%, at least 90%, at least 95%, at least 98%, at least 99%, or 100%, amino acid sequence identity to any Cas13a amino acid sequence, for example a Cas 13a sequence as shown in Table 1.

Additional Cas13 proteins include BzoCas13b (Bergeyvella zoohelcum; WP_002664492); PinCas13b (Prevotella intermedia; WP_036860899); PbuCas13b (Prevotella buccae; WP_004343973); AspCas13b (Alistipes sp. ZOR0009; WP_047447901); PsmCas13b (Prevotella sp.MA2016; WP_036929175); RanCas13b (Riemerella anatipestifer; WP_004919755); PauCas13b (Prevotella aurantiaca; WP_025000926); PsaCas13b (Prevotella saccharolytica, WP_051522484); Pin2Cas13b (Prevotella intermedia; WP_061868553); CcaCas13b (Capnocytophaga canimorsus; WP_013997271); PguCas13b (Porphyromonas gulae; WP_039434803); PspCasl3b (Prevotella sp. P5-125, WP_0440652940); PgiCasl3b (Porphyromonas gingivalis; WP_053444417); FbrCas13b (Flavobacterium branchiophilum; WP_014084666); and Pin3Cas13b (Prevotella intermedia; WP_050955369); FnsCas13c (Fusobacterium necrophorum subsp.funduliforme ATCC 51357contig00003: WP_005959231.1); FndCas13c (Fusobacterium necrophorum DJ-2 contig0065, whole genome shotgun sequence; WP_035906563.1); FnfCas13c (Fusobacterium necrophorum subsp. funduliforme 1_1_36S cont1.14; EHO19081.1); FpeCas13c (Fusobacterium perfoetens ATCC 29250 T364DRAFT _scaffold00009.9_C; WP_027128616.1); FulCas13c (Fusobacterium ulcerans ATCC 49185 cont2.38; WP_040490876.1); AspCas13c (Anaerosalibacter sp. ND1 genome assembly Anacrosalibacter massiliensis ND1; WP_042678931.1); Ruminococcus sp Cas13d, (GI: 1690532978); EsCas13d ([Eubacterium] siraeum DSM 15702; GI: 1486942132 or GI: 1486942131) and the Cas13d homologs disclosed in U.S. Pat. Publication No. 2019/0062724.

TABLE 1

Exemplary Cas13a proteins

| | | |
|---|---|---|
| Cas13a abbreviation | Organism name | Accession number |
| LshCas13a | Leptotrichia shahii | WP_018451595.1 |
| LwaCas13a | Leptotrichia wadei | WP_021746774.1 |
| LseCas13a | Listeria seeligeri | WP_012985477.1 |
| LbmCas13a | Lachnospiraceae bacterium MA2020 | WP_044921188.1 |
| LbnCas13a | Lachnospiraceae bacterium NK4A 179 | WP_022785443.1 |
| CamCas13a | [Clostridium] aminophilum DSM 10710 | WP_031473346.1 |
| CgaCas13a | Camobacterium gallinarum DSM 4847 | WP_034560163.1 |
| Cga2Cas13a | Camobacterium gallinarum DSM 4847 | WP_034563842.1 |
| Pprcas13a | Paludibacter propionicigenes WB4 | WP_013443710.1 |
| LweCas13a | Listeria weihenstephanensis FSL R9-0317 | WP_036059185.1 |
| LneCas13a | Listeriaceae bacterium FSL M6-0635 (Listeria newyorkensis) | WP_036091002.1 |
| Lwa2cas13a | Leptotrichia wadei F0279 | WP_021746774.1 |
| RcsCas13a | Rhodobacter capsulatus SB 1003 | WP_013067728.1 |
| RcrCas13a | Rhodobacter capsulatus R121 | WP_023911507.1 |
| RedCas13a | Rhodobacter capsulatus DE442 | WP_023911507.1 |
| LbuCas13a | Leptotrichia buccalis | WP_015770004.1 |
| LbaCas13a | Lachnospiraceae bacterium NK4A179 | WP_022785443.1 |
| RcaCas13a | Rhodobacter capsulatus R121 | ETD76934.1 |
| EreCasl3a | [Eubacterium] rectale | WP_055061018.1 |
| HheCas13a | Herbinix hemicellulosilytica | CRZ35554.1 |

In certain embodiments, the compositions, systems and methods include one or more Type V Cas proteins. Non-limiting examples of Type V CRISPR/Cas proteins include Cas12 and Cas14 proteins. See, e.g., U.S. Pat. Publication No. 2019/0241954. In some embodiments, the Cas12 protein is a Cas12 polypeptide comprising an amino acid sequence having at least 75%, at least 80%, at least 85%, at least 90%, at least 95%, at least 98%, at least 99%, or 100%, amino acid sequence identity to any Cas12 amino acid sequence. See e.g., International Patent Publication Nos. WO 2020/023529, WO 2019/104058, WO 2019/089796 and WO 2020/181101.

In some embodiments, the Cas14 protein is a Cas14 polypeptide comprising an amino acid sequence having at least 75%, at least 80%, at least 85%, at least 90%, at least 95%, at least 98%, at least 99%, or 100%, amino acid sequence identity to any Cas14 amino acid sequence. See e.g.. Harrington et al. (Harrington LB, (2018) Science 362(6416):839-842) and International Patent Publication No. WO 2020/181102 (Cas14).

In certain embodiments, additional detection system may comprise a Csm6 protein Csm6 is a family of single-stranded ribonucleic acid (ssRNA) endonucleases associated with Type III CRISPR-Cas systems. The RNA cleavage activity of Csm6 can be allosterically activated by binding of either cyclic oligoadenylates (cAₙ) or short linear oligoadenylates bearing a terminal 2′-3′ cyclic phosphate (Aₙ>P). Csm6 has been used in the SHERLOCK system to amplify the detection of viral RNAs. In some embodiments, EiCasm6 (Enterococcus italicus; WP_007208953.1), LsCsm6 (Lactobacillus salivarius; WP_081509150.1) and/or TtCsm6 (Thermus thermophilus: WP_011229148.1) is used.

In an embodiment, the Cas protein is a modified protein that is modified, or engineered or mutated, to alter its interaction with guide or target sequences and/or to alter its nuclease activity, for example specificity, turn-over, nucleotide preferences, etc. In other embodiments, the Cas protein may be fused to another protein, peptide, or marker to aid in isolation, identification, separation, nuclease activity, target sequence binding, etc.

In some cases, the additional nucleic acid detection systems detects RNA (viral RNA, mRNA, small RNAs, etc.) is directly detected (without the need for reverse transcriptase) using systems comprising wild-type and/or modified Cas13 proteins, while DNA (e.g.. amplicons generated from LAMP-based systems) is directly detected using systems comprising wild-type and/or engineered Cas12 or Cas14 proteins.

One or more of the same or different Cas effector proteins can be used. The one or more Cas proteins may themselves be the same or different (modified or engineered) proteins.

### Target Sequences

The target sequence can be from any source, including mammals, viruses, bacteria, and fungi. In some embodiments, the target sequence is a microbial or viral sequence, for example a coronavirus sequence such as SARS-CoV-2 (which causes COVID-19). In still other embodiments the target sequence is a mammalian genomic or transcribed sequence. In some embodiments, the source may be a human, non-human, or animal. In some embodiments, an animal source may be a domesticated or non-domestic animal, for example wild game. In some embodiments, the domesticated animal is a service or companion animal (e.g., a dog, cat, bird, fish, or reptile), or a domesticated farm animal.

For target sequences from pathogenic sources, the pathogen may have significant public health relevance, such as bacteria, fungus, or protozoan, and the target sequence may be found, without limitation, in one or more of coronavirus (e.g.. severe acute respiratory syndrome-related coronavirus (SARS), Middle East respiratory syndrome-related coronavirus (MERS), SARS-CoV-2, etc.), Hepatitis C virus, Japanese Encephalitis, Dengue fever, influenza virus or Zika virus. Any pathogen (e.g., virus, bacteria, etc.) can be detected. In some embodiments, the system described herein may be used to detect one or more pathogens.

A target sequence can be single stranded (ss) or double stranded (ds) DNA or RNA (e.g., viral RNA, mRNA, tRNA, rRNA, iRNA, miRNA, etc.). For LAMP-based systems, the target sequence may be converted from RNA (e.g., RNA virus) into DNA using any well-known method, for example, reverse transcriptase. Thus, the term “target sequence” includes DNA or RNA that has been converted into DNA.

In some cases, the target sequence is a viral sequence (e.g., a genomic RNA of an RNA virus or DNA of a DNA virus). As such, subject method can be for detecting the presence of a viral sequence amongst a population of nucleic acids (e.g., in a sample).

Non-limiting examples of possible RNA targets include viral RNAs such as coronavirus (SARS, MERS, SARS-CoV-2), Orthomyxoviruses, Hepatitis C Virus (HCV), Ebola disease, influenza, polio measles and retrovirus including adult Human T-cell lymphotropic virus type 1 (HTLV-1) and human immunodeficiency virus (HIV).

Non-limiting examples of possible target DNAs include, but are not limited to, viral DNAs such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus. Pityriasis Rosea, kaposi’s sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. In some cases, the target DNA is parasite DNA. In some cases, the target DNA is bacterial DNA. e.g., DNA of a pathogenic bacterium.

In some embodiments, the target nucleic acid is a DNA or RNA sequence associated with cancer. These can include genes that play a role in DNA methylation, histone modification, message splicing, and microRNA expression. Along with well-known examples such as the so-called Philadelphia chromosome associated with chronic myeloid leukemia, in some embodiments, the target is a DNA associated with a translocation such as t(8;14)(q24;q32), t(2;8)(p12;q24), t(8;22)(q24;q11), t(8;14)(q24;q11), and t(8;12)(q24;q22), each associated with an alteration of C-Myc and associated with acute lymphocytic leukemia. Other examples include t(10;14)(q24;q32) which effects the LYT10 gene and is associated with B cell lymphoma (see Nambiar (2008) Biochim Biophys Acta 1786:139-152). Other targets include mutant genes associated with cancers such as BRCA2 (ovarian cancer), BMP2, 3, 4, 7 (endometrial cancer), CAGE (cervical cancer), HOXA10 (ovarian cancer) and more (see Jeong et al. (2014) Front Oncol 4(12)).

In some cases, the methods and compositions of the invention are used to examine other disorders that display an altered transcriptional state. Examples include diabetes, metabolic syndrome (Hawkins et al. (2018) Peer J 6:e5062), Huntington syndrome and other neurological diseases (Xiang et al. (2018) Front Mol Neurosci 11:153) and cancer. In some cases, the methods and compositions are used to monitor response to a therapy administered for the treatment of a disorder characterized by an altered transcriptional state. In some cases, the methods and compositions are used to monitor altered transcriptional activity in a non-disease condition such as the onset of puberty, pregnancy or menopause.

### Samples

Any sample that includes nucleic acid (e.g., a plurality of nucleic acids) can be used in the compositions, systems and methods described herein. The term “plurality” is used herein to mean two or more. Thus, in some cases a sample includes two or more (e.g., 3 or more, 5 or more, 10 or more, 20 or more, 50 or more, 100 or more, 500 or more, 1.000 or more, or 5.000 or more) nucleic acids (e.g.. RNAs or DNAs). A subject method can be used as a very sensitive way to detect a target sequence present in a sample (e.g., in a complex mixture of nucleic acids such as RNAs or DNAs). In some cases, the sample includes 5 or more RNAs or DNAs (e.g., 10 or more, 20 or more, 50 or more, 100 or more, 500 or more, 1,000 or more, or 5,000 or more RNAs or DNAs) that differ from one another in sequence. In some cases, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, RNAs or DNAs. In some cases, the sample comprises from 10 to 20. from 20 to 50. from 50 to 100. from 100 to 500. from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁶ to 10⁶, from 10⁶ to 5×10⁶, or from 5×10⁶ to 10⁷, or more than 10⁷, RNAs or DNAs. In some cases, the sample comprises from 5 to 10⁷ RNAs or DNAs (e.g., that differ from one another in sequence) (e.g., from 5 to 10⁶, from 5 to 10⁵, from 5 to 50,000, from 5 to 30,000, from 10 to 10⁶, from 10 to 10.sup.5, from 10 to 50,000, from 10 to 30,000, from 20 to 10⁶, from 20 to 10⁵, from 20 to 50,000, or from 20 to 30,000 RNAs or DNAs). In some cases, the sample includes 20 or more RNAs or DNAs that differ from one another in sequence. In some cases, the sample includes RNAs or DNAs from a cell lysate (e.g., a eukaryotic cell lysate, a mammalian cell lysate, a human cell lysate, a prokaryotic cell lysate, a plant cell lysate, and the like). For example, in some cases the sample includes RNA or DNA from a cell such as a eukaryotic cell, e.g., a mammalian cell such as a human cell.

In any of the compositions and methods described herein, RNA from a sample may be reverse transcribed into DNA, for example for LAMP-based DNA amplification assays.

Suitable samples include but are not limited to saliva, blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed: or enrichment for certain cell populations, such as cancer cells. The definition also includes sample that have been enriched for particular types of molecules, e.g., DNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

In some embodiments, the sample is a saliva sample from a subject. Saliva sample collection can be performed with minimal contact between healthcase workers and subjects, improving safety and decreasing the need for personal protective equipment (PPE). Saliva sampling is reported to have 97% concordance with nasopharyngeal (NP) swab sampling in RT-qPCR detection, indicating that it can be a reliable method for detection of SARS-CoV-2 (Iwasake et al. (2020) J Infect 81(2):e145-e147 doi: 10.1016/j.jinf.2020.05.071, Wyllie et al. (2020)Medrxiv medrxiv.org/content/10.1101/2020.04.16.20067835v 1?fbclid=IwAR1sexAHAFxOuD iH7_7QKParCeIcotoL2DP8oMM27uKeV0bxE8d5IZYQXvM). Thus, in the context of the SARS-CoV-2 (and other respiratory disease-causing pathogens), the compositions, systems and/or methods described herein using saliva samples do not require RNA extraction and/or upper respiratory tract swabs, thereby minimizing the contact between health care workers and the subject.

The samples used in the systems, methods and/or compositions of the invention may also be subject to extraction, lysis (e.g. via enzymatic or high temperature treatments) and/or other treatments (e.g., chemical reduction, exposure to chaotropic agents. RNAse inhibitors, etc.). (Bloom et al. (2020) medRxiv: The Preprint Server for Health Sciences, September. doi.org/10.1101/2020.08.04.20167874.; Myhrvold et al. (2018) Science 360(6387):444-48). Alternatively, the systems, methods and/or compositions of the invention may be extraction-free (e.g., RNA extraction-free in the case of RNA viruses such as SARS-CoV-2).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell, tissue, or organ: a cell, tissue, or organ from an invertebrate animal (e.g., fruit fly, enidarian, echinoderm, nematode, an insect an arachnid, etc.); a cell, tissue, fluid, or organ from a vertebrate animal (e.g., fish, amphibian, reptile, bird, mammal); a cell, tissue, fluid, or organ from a mammal (e.g., a human: a non-human primate: an ungulate: a feline: a bovine; an ovine: a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria): fungus-like members of Protista, e.g., slime molds, water molds, etc.; animal-like members of Protista, e.g., flagellates (e.g., Euglena), amoeboids (e.g.. amoeba), sporozoans (e.g., Apicomplexa, Myxozoa, Microsporidia), and ciliates (e.g., Paramecium). Suitable sample sources include members of the kingdom Fungi, including, but not limited to, members of any of the phyla: Basidiomycota (club fungi; e.g., members of Agaricus, Amanita. Boletus, Cantherellus, etc.); Ascomycota (sac fungi, including, e.g., Saccharomyces); Mycophycophyta (lichens); Zygomycota (conjugation fungi); and Deuteromycota. Suitable sample sources include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include members of the kingdom Animalia, including, but not limited to, members of any of the following phyla: Porifera (sponges); Placozoa; Orthonectida (parasites of marine invertebrates); Rhombozoa: Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida: Kinorhyncha; Loricifera; Acanthocephala; Entoprocta; Nemotoda; Nematomorpha; Cycliophora: Mollusca (mollusks); Sipuncula (peanut worms); Annelida (segmented worms); Tardigrada (water bears); Onychophora (velvet worms); Arthropoda (including the subphyla: Chelicerata. Myriapoda. Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g., starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea): Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Ayes (birds); and Mammalian (mammals) Suitable plants include any monocotyledon and any dicotyledon.

Samples may be collected from the environment. For example, a sample may be taken from a surface by a swab (e.g., “high touch” surfaces such as hospital door knobs, light switches and the like (WHO /2019-nCoV/Environmental _protocol/2020.1). Other examples include air sampling and water sampling from a facility’s distribution system. Environmental samples may also be collected from sewage and waste water treatment plants (Hellmer et al. (2014) Appl Envir Microbiol 80(21):6771- 6781; Mallapaty (2020) Nature 580:176-177).

Suitable sources of a sample include cells, fluid, tissue, or organ taken from an organism; from a particular cell or group of cells isolated from an organism: etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some cases, the source of the sample is a (or is suspected of being a diseased cell, fluid, tissue, or organ, for example of a human subject. In some cases, the source of the sample is a normal (non-diseased) cell, fluid, tissue, or organ. In some cases, the source of the sample is a (or is suspected of being a pathogen-infected cell, tissue, or organ. For example, the source of a sample can be an individual who may or may not be infected--and the sample could be any biological sample (e.g.. blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some cases, the sample is a cell-free liquid sample. In some cases, the sample is a liquid sample that can comprise cells.

Pathogens to be detected in samples include viruses, bacteria, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga’s disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., coronaviruses (e.g.. COVID-19, MERS, SARS, etc.); immunodeficiency virus (e.g., HIV); influenza virus; dengue; West Nile virus: herpes virus; yellow fever virus; Hepatitis Virus C: Hepatitis Virus A: Hepatitis Virus B: papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi’s sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi’s sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40. mouse mammary tumor virus, dengue virus, rubella virus, West Nile virus, Plasmodium falciparum, Plasmodium vivax, Toxoplasma gondii, Trypanosoma rangeli, Trypanosoma cruzi. Trypanosoma rhodesiense, Trypanosoma brucei, Schistosoma mansoni, Schistosoma japonicum, Babesia bovis. Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orate, M. arginini, Acholeplasma laidlawii, M. salivarium and M. pneumoniae.

### Reporters

Any reporter molecule (also referred to as a “detectable label” or “signal moiety”) can be used in the LAMP-based and/or additional nucleic acid detection systems described herein, including but not limited to, CRISPR-Cas detection systems, molecular beacons, FRET probes, split-fluorescent protein probes, RNA aptamers, fluorescent labels, enzymatic labels and/or bioluminescent labels.

In some cases, the signal moiety is a fluorescent label. Examples of fluorescent labels include, but are not limited to: an Alexa Fluor™. dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B. ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3. Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red. Oregon Green, Pacific Blue, Pacific Green, Pacific Orange, quantum dots, and a tethered fluorescent protein.

In some cases, a detectable label is a fluorescent label selected from: an Alexa Fluor™. dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rhol4. ATTO 633. ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange.

In some cases, a detectable label is a fluorescent label selected from: an Alexa Fluor™ dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B. ATTO Rho11, ATTO Rho12, ATTO Thio12, ATTO Rho101, ATTO 590, ATTO 594, ATTO Rho13, ATTO 610, ATTO 620. ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5. Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

Examples of ATTO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor™ 350, Alexa Fluor™ 405, Alexa Fluor™ 430, Alexa Fluor™ 488. Alexa Fluor™ 500. Alexa Fluor™ 514, Alexa Fluor™ 532, Alexa Fluor™ 546, Alexa Fluor™ 555, Alexa Fluor™ 568, Alexa Fluor™ 594, Alexa Fluor™ 610, Alexa Fluor™ 633, Alexa Fluor™ 635, Alexa Fluor™ 647, Alexa Fluor™ 660, Alexa Fluor™ 680, Alexa Fluor™ 700, Alexa Fluor™ 750, Alexa Fluor™ 790, and the like.

In some cases, cleavage of a labeled detector can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ration of one color to another, and the like.

In some cases, signal is detected using lateral flow chromatography. In a simple sandwich type of system, the sample is applied to a pad in the lateral flow device that acts as the first stage of the absorption process, and in some cases contains a filter, to ensure the accurate and controlled flow of the sample. The conjugate pad, which stores the conjugated labels and antibodies, will receive the sample. If the target is present, the immobilized conjugated antibodies and labels will bind to the target and continue to migrate along the test. As the sample moves along the device the binding reagents situated on the nitrocellulose membrane will bind to the target at the test line. A colored line will form and the density of the line will vary depending on the quantity of the target present. Some targets may require quantification to determine target concentration. This is where a rapid test can be combined with a reader to provide quantitative results.

In some cases, the methods are carried out with a reporter molecule that is detected via lateral flow. In some cases, Milenia Genline HybriDetect 1 (TwistDx™) dipsticks are used. For example, in some cases the step of measuring can include one or more of: gold nanoparticle-based detection (e.g., see Xu et al. (2017) Angew Chem Int Ed Engl. 46(19):3468-70; and Xia et al. (2010) Proc Natl Acad Sci U S A. Jun 15: 107(24):10837-41), fluorescence polarization, colloid phase transition/dispersion (e.g., Baksh et al. (2004) Nature. Jan 8; 427(6970): 139-41), electrochemical detection, semiconductor- based sensing (e.g., Rothberg et al. (2011) Nature Jul 20; 475(7356):348-52; e.g., one could use a phosphatase to generate a pH change after ssDNA cleavage reactions, by opening 2′ -3′ cyclic phosphates, and by releasing inorganic phosphate into solution), and detection of a labeled detector ssDNA. The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor-based detection of the presence or absence of a color (i.e., color detection method), and the presence or absence of (or a particular amount of) an electrical signal.

In some cases, the detectable signal that is measured is produced by the fluorescence -emitting dye pair. For example, in some cases, a subject method includes contacting a sample with a labeled detector ssDNA comprising a fluorescence resonance energy transfer (FRET) pair or a quencher/fluor pair, or both.

In some cases, a subject method includes contacting a sample with a labeled detector ssDNA comprising a FRET pair. In some cases, a subject method includes contacting a sample with a labeled detector ssDNA comprising a fluor/quencher pair. Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.” In some cases (e.g., when the detector ssDNA includes a FRET pair) the labeled detector ssDNA produces an amount of detectable signal prior to being cleaved, and the amount of detectable signal that is measured is reduced when the labeled detector ssDNA is cleaved. In some cases, the labeled detector ssDNA produces a first detectable signal prior to being cleaved (e.g., from a FRET pair) and a second detectable signal when the labeled detector ssDNA is cleaved (e.g., from a quencher/fluor pair). As such, in some cases, the labeled detector ssDNA comprises a FRET pair and a quencher/fluor pair. In some cases, the labeled detector ssDNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Forster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”. The donor-acceptor pair (a FRET donor moiety and a FRET acceptor moiety) is referred to herein as a “FRET pair” or a “signal FRET pair.” Thus, in some cases, a subject labeled detector ssDNA includes two signal partners (a signal pair), when one signal partner is a FRET donor moiety and the other signal partner is a FRET acceptor moiety. A subject labeled detector ssDNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated. FRET donor and acceptor moieties (FRET pairs) will be known to one of ordinary skill in the art and any convenient FRET pair (e.g., any convenient donor and acceptor moiety pair) can be used. See: Bajar et al. (2016) Sensors (Basel). Sep 14; 16(9); and Abraham et al. (2015) PLoS One Aug 3; 10(8):e0134436.

### Methods

Thus, methods of the invention include (a) contacting a sample potentially including the target sequence with: (i) any of the compositions or systems as described herein to generate amplicons representing the target sequence; and (b) measuring a detectable signal of the amplicons, thereby detecting the target sequence (DNA or RNA) in the sample. In still further aspects, the methods comprise: (a) contacting a sample potentially including the target sequence with: (i) any of the compositions or systems as described herein to generate amplicons representing the target sequence and convert the amplicons to RNA; and (b) measuring a detectable signal of the amplicons, thereby detecting the target sequence in the sample. In certain embodiments, step (b) comprises detecting the amplicons (including RNA amplicons) using one or more additional nucleic acid detection systems (e.g., CRISPR based systems).

In some cases, the methods comprise: obtaining nucleic acids (target sequence) from a sample, optionally wherein RNA target sequences are converted to DNA target sequences; subjecting the nucleic acid target sequences to any of the LAMP-based systems described here, thereby amplifying the target sequences (generating LAMP amplicons also referred to as LAMPlicons), optionally wherein DNA amplicons are converted to RNA. In certain embodiments, the amplicons are directly detected. In other embodiments, the methods further comprise subjecting the DNA or RNA amplicons to one or more additional nucleic acid detection systems, thereby detecting the target sequence in the sample. The methods may also comprise measuring the detectable label and, optionally quantifying the levels.

The contacting steps and measuring steps may be performed in the same or different containers and in liquid and/or solid supports. For example, the contacting may be performed in the same container and transferred for detection or, alternatively, the contacting and measuring steps may be performed in the same container.

The assay mixture may be incubated under various conditions to allow a target nucleic acid sequence, if present in the sample, to hybridize to the LAMP primers. In some embodiments, the conditions are designed to aid in hybridization, wherein the sequences are 100% complementary. In other embodiments, the conditions for incubation of the assay mixture may be varied to allow for less than 100% complementarity between the guide RNA sequence and the target sequence, for example 1 mismatch between target nucleic acid and guide RNA, or less than about 2 mismatches, 3 mismatches, 4 mismatches, or 5 mismatches. See, e.g., Zhou et al., supra.

The contacting step of a subject methods can be carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo.

The contacting step may be for any length of time, including but not limited to 2 hours or less (e.g., 1.5 hours or less, 1 hour or less. 40 minutes or less, 30 minutes or less, 20 minutes or less, 10 minutes or less, or 5 minutes or less, or 1 minute or less) prior to the measuring step. For example, in some cases the sample is contacted for 40 minutes or less prior to the measuring step. In some cases, the sample is contacted for 20 minutes or less prior to the measuring step. In some cases, the sample is contacted for 10 minutes or less prior to the measuring step. In some cases, the sample is contacted for 5 minutes or less prior to the measuring step. In some cases, the sample is contacted for 1 minute or less prior to the measuring step. In some cases, the sample is contacted for from 50 seconds to 60 seconds prior to the measuring step. In some cases, the sample is contacted for from 40 seconds to 50 seconds prior to the measuring step. In some cases, the sample is contacted for from 30 seconds to 40 seconds prior to the measuring step. In some cases, the sample is contacted for from 20 seconds to 30 seconds prior to the measuring step. In some cases, the sample is contacted for from 10 seconds to 20 seconds prior to the measuring step. In some embodiments, the sample is incubated with the Cas protein for less than about 2 hrs., 90 min., 60 min., 40 min., 30 min., 20 min., 10 min., 5 min., 4 min., 3 min., 2 min., 1 min.. 55 sec., 50 sec., 40 sec., 30 sec.. 20 sec., or 10 sec., and more than about 5 sec., 10 sec., 20 sec., 30 sec., 40 sec., 50 sec., 60 sec., 2 min., 3 min., 4 min., 5 min., 10 min., 20 min., 30 min., 40 min., 50 min., 60 min., or 90 min.

The method may be conducted at any suitable temperature. LAMP-based assays (methods) are typically conducted at 60-65° C. In some embodiments, the assays, for example the optional additional nucleic acid detection methods are conducted at a physiological temperature, for example about 37° C. This allows the methods to be readily practiced in any location, including a doctor’s office or home (for example by performing the assay using body temperature (e.g., holding the assay contained under the arm, against the skin, etc.).

The methods described herein can detect the target sequence (RNA or DNA) with a high degree of sensitivity. In some cases, a method of the present disclosure can be used to detect a target sequence present in a sample comprising a plurality of nucleotides (including the target sequence and a plurality of non-target sequences), where the target sequence is present at one or more copies per 10⁷ non-target sequences (e.g., one or more copies per 10⁶ non-target sequences, one or more copies per 10⁵ non-target sequences, one or more copies per 10⁴ non-target sequences, one or more copies per 10³ non-target sequences, one or more copies per 10² non-target sequences, one or more copies per 50 non-target sequences, one or more copies per 20 non-target sequences, one or more copies per 10 non-target sequences, or one or more copies per 5 non-target sequences). In some cases, a method of the present disclosure can be used to detect a target sequences present in a sample comprising a plurality of sequences (including the target sequences and a plurality of non-target sequences), where the target sequence is present at one or more copies per 10¹⁸ non-target sequences (e.g., one or more copies per 10¹⁵ non-target sequences, one or more copies per 10¹² non-target sequences, one or more copies per 10⁹ non-target sequences, one or more copies per 10⁶ non-target sequences, one or more copies per 10⁵ non-target sequences, one or more copies per 10⁴ non-target sequences, one or more copies per 10³ non-target sequences, one or more copies per 10² non-target sequences, one or more copies per 50 non-target sequences, one or more copies per 20 non-target sequences, one or more copies per 10 non-target sequences, or one or more copies per 5 non-target sequences).

In some cases, a method of the present disclosure can detect a target sequence present in a sample, where the target sequences is present at from one copy per 10⁷ non-target sequences to one copy per 10 non-target sequences (e.g., from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per ¹⁰⁷ non-target sequences to 1 copy per 10⁶ non-target sequences, from 1 copy per 10⁶ non-target sequences to 1 copy per 10 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 10 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, a method of the present disclosure can detect a target sequence (RNA or DNA) present in a sample, where the target sequences is present at from one copy per 10¹⁸ non-target sequences to one copy per 10 non-target sequences (e.g., from 1 copy per 10¹⁸ non-target sequences to 1 copy per 10² non-target sequences, from 1 copy per 10¹⁵ non-target sequences to 1 copy per 10² non-target sequences, from 1 copy per 10¹² non-target sequences to 1 copy per 10² non-target sequences, from 1 copy per 10⁹ non-target sequences to 1 copy per 10² non-target sequences, from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per 10⁷ non-target sequences to 1 copy per 10⁶ non-target sequences, from 1 copy per 10⁶ non-target sequences to 1 copy per 10 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 10 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, a method of the present disclosure can detect a target sequence present in a sample, where the target sequence is present at from one copy per 10⁷ non-target sequences to one copy per 100 non-target sequences (e.g., from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per 10⁷ non-target sequences to 1 copy per 10⁶ non-target sequences, from 1 copy per 10⁶ non-target sequences to 1 copy per 100 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 100 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, the threshold of detection, for a subject method of detecting a target sequence in a sample, is 10 nM or less. The term “threshold of detection” is used herein to describe the minimal amount of target sequence that must be present in a sample in order for detection to occur. Thus, as an illustrative example, when a threshold of detection is 10 nM, then a signal can be detected when a target sequence is present in the sample at a concentration of 10 nM or more. In some cases, a method of the present disclosure has a threshold of detection of 5 nM or less. In some cases, a method of the present disclosure has a threshold of detection of 1 nM or less. In some cases, a method of the present disclosure has a threshold of detection of 0.5 nM or less. In some cases, a method of the present disclosure has a threshold of detection of 0.1 nM or less. In some cases, a method of the present disclosure has a threshold of detection of 0.05 nM or less. In some cases, a method of the present disclosure has a threshold of detection of 0.01 nM or less. In some cases, a method of the present disclosure has a threshold of detection of 0.005 nM or less. In some cases, a method of the present disclosure has a threshold of detection of 0.001 nM or less. In some cases, a method of the present disclosure has a threshold of detection of 0.0005 nM or less. In some cases, a method of the present disclosure has a threshold of detection of 0.0001 nM or less. In some cases, a method of the present disclosure has a threshold of detection of 0.00005 nM or less. In some cases, a method of the present disclosure has a threshold of detection of 0.00001 nM or less. In some cases, a method of the present disclosure has a threshold of detection of 10 pM or less. In some cases, a method of the present disclosure has a threshold of detection of 1 pM or less. In some cases, a method of the present disclosure has a threshold of detection of 500 fM or less. In some cases, a method of the present disclosure has a threshold of detection of 250 fM or less. In some cases, a method of the present disclosure has a threshold of detection of 100 fM or less. In some cases, a method of the present disclosure has a threshold of detection of 50 fM or less. In some cases, a method of the present disclosure has a threshold of detection of 500 aM (attomolar) or less. In some cases, a method of the present disclosure has a threshold of detection of 250 aM or less. In some cases, a method of the present disclosure has a threshold of detection of 100 aM or less. In some cases, a method of the present disclosure has a threshold of detection of 50 aM or less. In some cases, a method of the present disclosure has a threshold of detection of 20 aM or less. In some cases, a method of the present disclosure has a threshold of detection of 10 aM or less. In some cases, a method of the present disclosure has a threshold of detection of 1 aM or less.

In some cases, the threshold of detection (for detecting the target sequence in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target sequence at which the target sequence can be detected). In some cases, a method of the present disclosure has a threshold of detection in a range of from 800 fM to 100 pM. In some cases, a method of the present disclosure has a threshold of detection in a range of from 1 pM to 10 pM. In some cases, a method of the present disclosure has a threshold of detection in a range of from 10 fM to 500 fM, e.g., from 10 fM to 50 fM, from 50 fM to 100 fM, from 100 fM to 250 fM. or from 250 fM to 500 fM.

In some cases, the minimum concentration at which a target sequence (DNA or RNA) can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM. from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target sequence can be detected in a sample is in a range of from 800 fM to 100 pM. In some cases, the minimum concentration at which a target sequence can be detected in a sample is in a range of from 1 pM to 10 pM.

In some cases, the threshold of detection (for detecting the target sequences), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM. from 500 aM to 1 nM, from 500 aM to 500 pM, from 500 aM to 200 pM, from 500 aM to 100 pM. from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM. from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target sequence at which the target sequence can be detected). In some cases, a method of the present disclosure has a threshold of detection in a range of from 1 aM to 800 aM. In some cases, a method of the present disclosure has a threshold of detection in a range of from 50 aM to 1 pM. In some cases, a method of the present disclosure has a threshold of detection in a range of from 50 aM to 500 fM.

In some cases, the minimum concentration at which a target sequence can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM. from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM. from 500 aM to 1 pM, from 750 aM to 1 nM. from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM. from 1 pM to 1 nM, from 1 pM to 500 pM, from 1 pM to 200 pM, from 1 pM to 100 pM, or from 1 pM to 10 pM). In some cases, the minimum concentration at which a target sequence can be detected in a sample is in a range of from 1 aM to 500 pM. In some cases, the minimum concentration at which a target sequence can be detected in a sample is in a range of from 100 aM to 500 pM.

In some cases, a subject composition or method exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

The measuring can in some cases be quantitative, e.g., in the sense that the amount of signal detected can be used to determine the amount of target sequence present in the sample. The measuring can in some cases be qualitative, e.g., in the sense that the presence or absence of detectable signal can indicate the presence or absence of targeted sequence (e.g., virus, SNP, etc.). In some cases, a detectable signal will not be present (e.g., above a given threshold level) unless the targeted sequences(s) (e.g., virus, SNP, etc.) is present above a particular threshold concentration. As such, for example, as would be understood by one of ordinary skill in the art, a number of controls can be used if desired in order to set up one or more reactions, each set up to detect a different threshold level of target sequence, and thus such a series of reactions could be used to determine the amount of target sequence present in a sample (e.g., one could use such a series of reactions to determine that a target sequence is present in the sample ‘at a concentration of at least X’).

In some cases, a method of the present disclosure can be used to determine the amount of a target sequence in a sample (e.g., a sample comprising the target sequence and a plurality of non-target sequences). Determining the amount of a target sequence in a sample can comprise comparing the amount of detectable signal generated from a test sample to the amount of detectable signal generated from a reference sample. Determining the amount of a target sequence in a sample can comprise: measuring the detectable signal to generate a test measurement; measuring a detectable signal produced by a reference sample to generate a reference measurement and comparing the test measurement to the reference measurement to determine an amount of target sequence present in the sample.

RNase inhibitors may be used in the methods as described herein. In some embodiments, the assay mixture may include one or more molecules that inhibit non-Cas13a-dependent RNase activity, but do not affect RNase activity by activated Cas13a proteins. For example, the inhibitor may inhibit mammalian, bacterial, or viral RNases, such as, without limitation, RNase A and RNase H. In some embodiments, the RNase Inhibitor may be added to the sample to help preserve a target nucleic acid sequence. In these embodiments, the method may include a step of adding one or more RNA preserving compounds to the sample, for example one or more RNase inhibitors.

Detecting the label may be achieved in various ways known in the art. For example, detection of colorimetric, fluorescent, or luminescent labels may be accomplished by measurement of absorbance or emission of light at a particular wavelength. In some embodiments the signal may be detected by visual inspection, microscope, or light detector.

### Kits

The present disclosure provides a kit for detecting a target nucleotide sequences, e.g., in a sample comprising a plurality of sequences. In some cases, the kit comprises one or more LAMP-based compositions or systems as described herein. Positive and/or negative controls may also be included and/or instructions for use may also be included.

### EXAMPLES

### Materials and Methods

### CRISPR-Cas Direct Nucleic Acid Detection

For Lachnospiraceae bacterium ND2006 Cas12a (LbCas12a), crRNA and the activatorDNA were synthesized via Integrated DNA Technologies (IDT). Detection with Cas12a was performed using 1X of NEB 2.1 buffer (B7202S), 100 nM of LbCas12a protein, 100 nM crRNA, varying concentration (100 nM, 100 nM, 10 nM, 1 nM, 100 pM. 10 pM) of dsDNA activator, and 200 nM of Dnase Alert (IDT). LbCas12a and crRNA was pre-mixed at 10X concentration in 1:1 molar ratio and incubated at RT for 15 minutes before adding it to the reaction. The reaction was heated at 37° C. with 15 sec interval detection with a Tecan Spark multimode microplate reader. For background subtraction, the fluorescence values of the no-activator reaction were subtracted from the fluorescence values of sample reactions at each time point. Maximum fluorescence was obtained by mixing DNase 1 and 200 nM DNaseAlert with 1X NEB 2.1 buffer. This saturated maximum fluorescence value was divided in half, time to reach this half-maximum fluorescence was calculated for each concentration of activator.

For Leptotrichia buccalis Cas13a (LbuCas13a), the crRNA and the activator were commercially synthesized from Synthego. Detection with Cas 13a was performed using 1X of Cas13 buffer, 100 nM of LbuCas 13a protein, 100 nM crRNA, varying concentrations (100 nM, 100 nM, 10 nM, 1 nM, 100 pM, 10 pM) of ssRNA, and 200 nM of Cas13 reporter (IDT). LbuCas13a and crRNA was premixed at 10X concentration in 1:1 ratio and incubated at room temperature for 15 minutes before adding it to the reaction. The rest of the reaction, and time to half-maximum fluorescence, was performed or analyzed as described above. Maximum fluorescence was obtained by mixing RNase A and 200 nM Cas13 reporter with 1X buffer. 5X Cas13 buffer (pH 6.8) is composed of 100 mM HEPES-Na pH 6.8 (Sigma), 250 mM KCl (Sigma), 25 mM MgCl2 (Sigma), and 25% glycerol (Thermo Fisher).

### LbuCas13 Protein Production

The expression and purification of Leptotrichia buccalis Cas13a (LbuCas 13a) was performed as previously described (East-Seletsky et al. (2017) Mol Cell 66(3):373-83.e3) with modifications, summarized here: His6-MBP-TEV-tagged Cas13a was prepared in E. coli Rosetta2 (DE3) grown in TB at 37° C. At an OD600 of 0.6-0.8. cultures were cooled on ice for 15 minutes prior to induction with 0.5 mM isopropyl β-D-1-thiogalactopyranoside (IPTG) and expression overnight at 16° C. Cell pellets measuring close to 10 mL in a 50 mL falcon tube were resuspended in 100 mL of Lysis buffer each. Soluble His6-MBP-TEV Cas13a was clarified by centrifugation at 15,000 g, then loaded onto a 5 mL HiTrap NiNTA column (GE Healthcare) and eluted over a linear imidazole (0.01- 0.3 M) gradient via FPLC (ÄKTA Pure). Following overnight dialysis and TEV digestion. LbuCas13a underwent purification by ion exchange and removal of MBP (5 mL HiTrap SP, MBP trap HP, GE Healthcare). Finally, size-exclusion chromatography was performed (S200 16/60, GE Healthcare) with 1 mM TCEP supplemented into the gel filtration buffer and peak fractions were pooled, concentrated and aliquoted into PCR strip tubes then snap frozen in LN2.

### LbCas12a Protein Production

The expression and purification of Lachnospiraceae bacterium ND2006 Cas12a (LbCas12a) was performed as previously described with the addition of 1 mM EDTA in the dialysis buffer (Knott et al. (2019) Nat Struct Mol Biol 26(4):315-21).

### LAMP and rLAMP Reactions

LAMP and rLAMP DNA amplification reactions were conducted with 2X LAMP Mastennix (NEB. E1700), 0.4 ul LAMP dye (NEB. E1700), 2 ul of 10X primer mix (IDT: 16 uM of FIP/BIP: 4 uM Floop/Bloop: 2 uM of F3B3), and 7.6 ul of sample. 8 ul of sample was added in reactions in which no dye was added. Detection was performed at 65° C. for 30 - 60 min in a CFX96 Touch Real-Time PCR machine (Bio-Rad). Time to threshold was calculated using single threshold analysis mode. Limit of detection for LAMP was determined as the concentration where there was amplification observed with at least 95% of the samples.

Twist Synthetic SARS-CoV-2 RNA Control 2 (Twist Bioscience. SKU 102024) was used as a template to perform the primer screen and establish LOD ranges. The volume of template to be used and the final concentration of template in the reaction was calculated based on the initial concentration provided by the vendor as 10^{∧}6 cp/ul.

### LAMP Assay Development

Restriction digestion of rLAMP DNA products was performed on 3 ul of amplified rLAMP product using 2 ul of AfeI (NEB R0652L), 2 ul 10X CutSmart Buffer, and 13 ul of water. This 20 ul of reaction was heated at 37° C. for 30 minutes. Undigested and digested rLAMP products were treated with 4 ul 1 u/ul RQ DNase I and 1 ul of 10X reaction buffer (Promega, M6101). The reaction was heated at 37° C. for 30 minutes, 1 ul of STOP solution (Promega M6101) was added, and incubated at 65° C. for 10 minutes, rLAMP products were analyzed using 15% TBE-Urea gels (Thermo Fisher, EC68855BOX).

### Direct Saliva Lysis

SARS-CoV-2 negative saliva (Lee Biosolutions, 991-05-S-25) was used to check compatibility of saliva with the DISCoVER workflow. All saliva samples were collected prior to November 2019 as per vendor. When viral synthetic RNA was used. Twist Synthetic SARS-CoV-2 RNA Control 2 was spiked in the commercial saliva and used as a template for mock samples. For limit-of-detection determination. SARS-CoV-2 viral seedstocks were spiked in negative saliva background under BSL-3 containment and used as a template for mock samples.

Commercial saliva was mixed with lysis reagents and heated at 75° C. for 30 minutes using a heat block. Twist synthetic RNA was added after completion of heat inactivation of saliva. Viral seed stocks were spiked in saliva in BSL3 facility prior to heat inactivation. Inactivating Reagent 1 was 2.5 mM TCEP /ImM EDTA at 1X concentration when mixed with saliva. Inactivating Reagent 2 was 100 mM TCEP/1mM EDTA at 1X concentration when mixed with saliva. Inactivating Reagent 3 was Quickextract DNA (Lucigen QE09050), Inactivating Reagent 4 was Quickextract RNA (Lucigen QER090150), and Inactivating Reagent 5 was RNA/DNAShield (Zymo 76020-420).

### Limit of Detection (LOD) Determination

To determine the LOD. SARS-CoV-2 viral seedstock was diluted in media to obtain various concentrations, spiked into saliva, and inactivated by adding 1X of Low TCEP/EDTA inactivating reagent and heating the samples at 75° C. The dilutions of virus in media were inactivated by heating at 75° C. for 30 minutes to comply with EH&S requirements (UC Berkeley) and RT-qPCR was performed using Luna® Universal Probe One-Step RT-qPCR Kit (NEB E3006) and E gene-targeting primers to determine the final concentration of viral RNA. Synthetic genomic RNA fragments (Twist Synthetic SARS-CoV-2 RNA Control 2) were used to obtain a standard curve for the calculations. The mock sample was detected as positive if the fold change (ratio of fluorescence value of sample to fluorescence value of no template control) is greater than 5 within 5 minutes. LOD for the mock samples was determined as the concentration where there was detection with at least 95% of the samples.

### Cas13a Guide Design Pipeline

LbuCas13a guides were designed according to their sensitivity, specificity, predicted secondary structure, and secondary structure of the viral target region. 59,760 candidate spacers were designed using the SARS-CoV-2 reference genome (wuhCor1), targeting both the forward and reverse strands. Sensitivity was determined by pairwise aligning available SARS-CoV-2 genomes with the reference and calculating the number of mismatches between the sample and reference genome for each candidate spacer. The percentage of genomes detected by each spacer is then calculated, allowing for no more than one mismatch. To ensure specificity, candidate spacers that were determined to be sensitive were aligned to other reference genomes of human coronaviruses, specifically, SARS, MERS, 229E. NL63, OC43 and HKU1. The percentage of missed genomes is then calculated, allowing for no more than two mismatches. These were also aligned to the human transcriptome, again allowing for two mismatches, to ensure these guides are not complementary to off-target human transcripts. Complementarity to off-target pathogens was also determined by searching for matches and calculating Hamming distance of each candidate spacer in NCBI viral genomes, NCBI bacterial genomes, and metagenomic samples from nasopharyngeal and oropharyngeal, adult saliva, and pediatric salvia samples. To ensure that the spacer sequences will allow for Cas13a binding to the direct repeat scaffold, the concatenated repeat and spacer sequence is folded using RNAfold, and evaluated for correct hairpin structure in the direct repeat and single strandedness in the spacer sequence. Lastly, the quality of the viral target is determined by prioritizing regions of persistent single-strandedness. Single-stranded regions were determined using SHAPE-MaP, and DMS-MapSeq was used to determine unstructured regions, and guides targeting in these regions were prioritized for screening.

### T7 Transcription

Transcription was performed on amplified rLAMP product using 2 ul of 1 mg/ml of T7 polymerase, 4 ul of 100 mM NTP mix (NEB N0450), 1ul of 200 mM DTT, 4 ul of 5X transcription buffer, 2 ul of LAMP product and 7 ul of water. This 20 ul of reaction was heated at 37° C. for 30 minutes. 5X transcription buffer is composed of 150 mM Tris-Cl, pH 8.1 at RT, 125 mM MgCl2, 0.05% Triton X-100 (Sigma Aldrich, X100), and 10 mM spermidine and stored at -20° C.

### Cas13 Detection

Cas13 detection was performed as a 20 ul reaction using 2 ul of 1:100 diluted transcription product of LAMP, 1 ul 5X Cas13 buffer, 2 ul of 2 uM Cas13 reporter, 2 ul of 10X RNP, 13 ul of 1X Cas13 buffer. 10X RNP was made as a 2:1 ratio of Cas13:crRNA with final concentration of Cas13 as 20 nM and incubated at RT for 15 minutes before adding it to the reaction. This 20 ul reaction was heated at 37° C. and read every 30 s in TECAN Spark multimode microplate reader using FAM channel (Excitation:485; Emission:535).

### One-pot T7 Transcription and Cas13 Detection

One pot T7 and Cas13 was performed by combining 2 ul of 1 mg/ml of T7 polymerase, 20 mM NTP mix, 10 mM DTT, and 25 mM MgCl2 with the Cas13 reaction mix. 2 ul of 1:100 dilution of rLAMP product is used for 20 ul of one pot T7-Cas13 reaction.

### Example 1: Amplification of a Target RNA

To amplify a target RNA of interest, a combination of coordinated amplification steps were carried out.

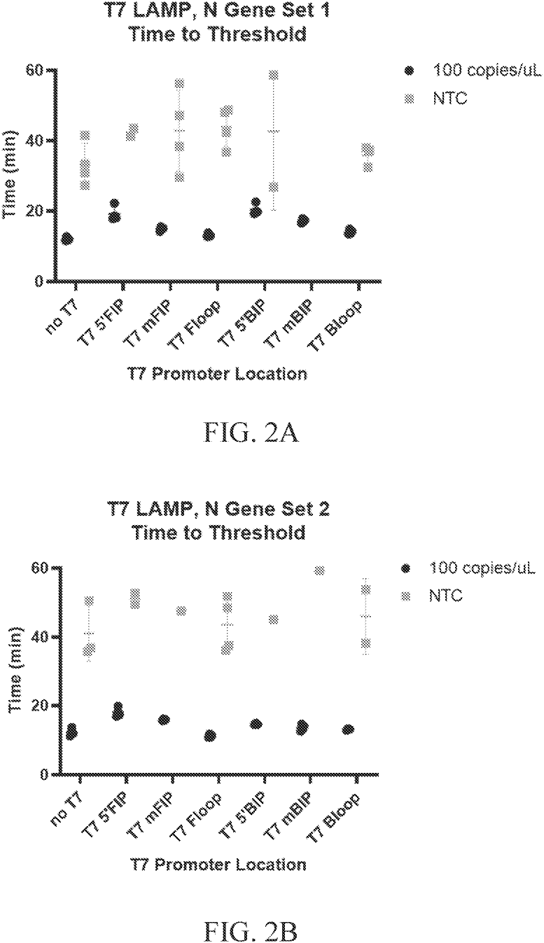

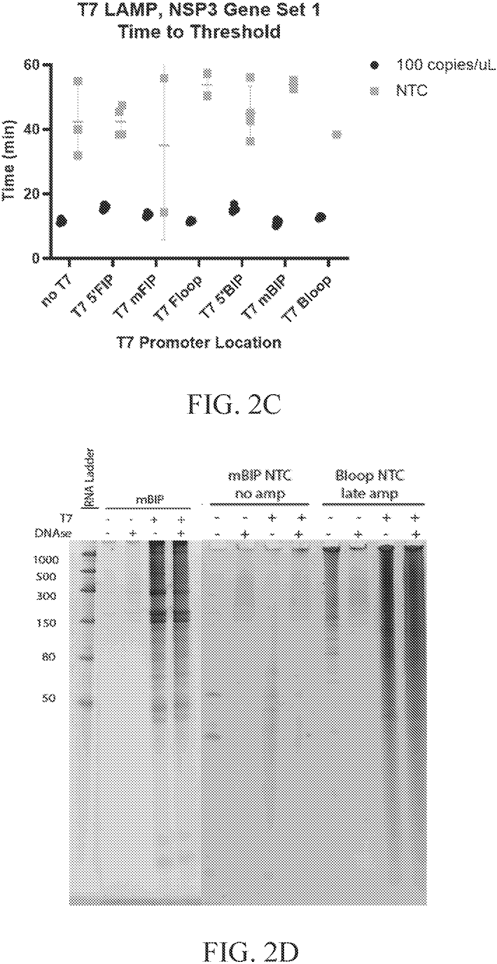

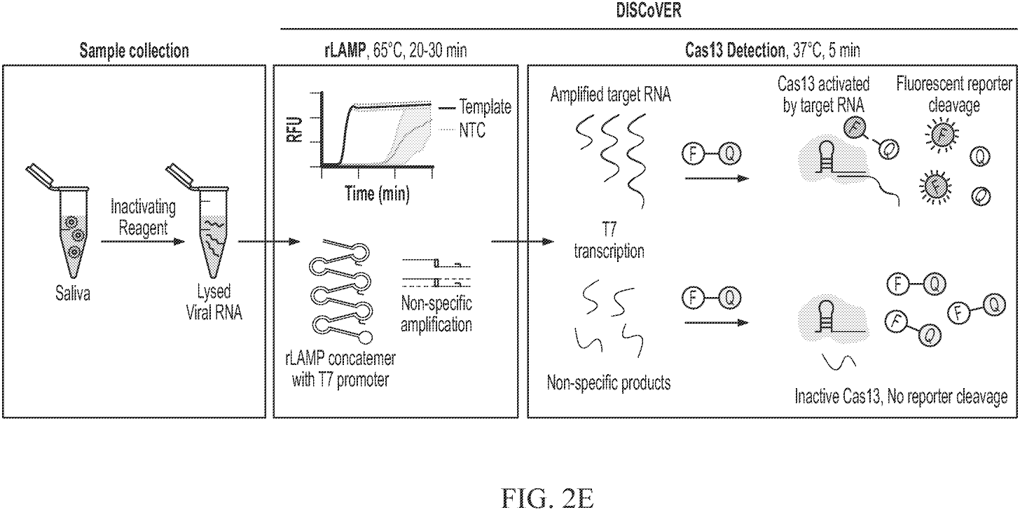

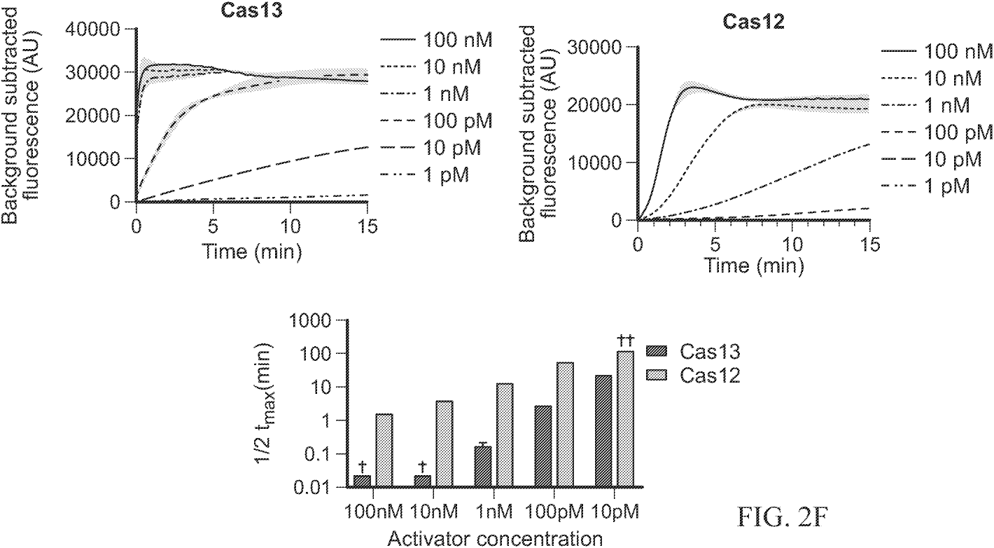

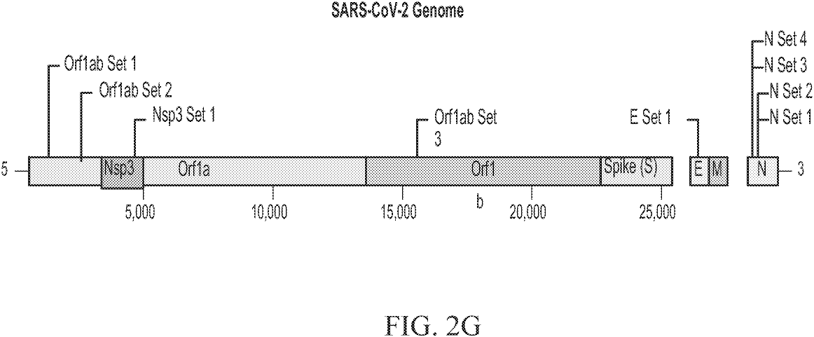

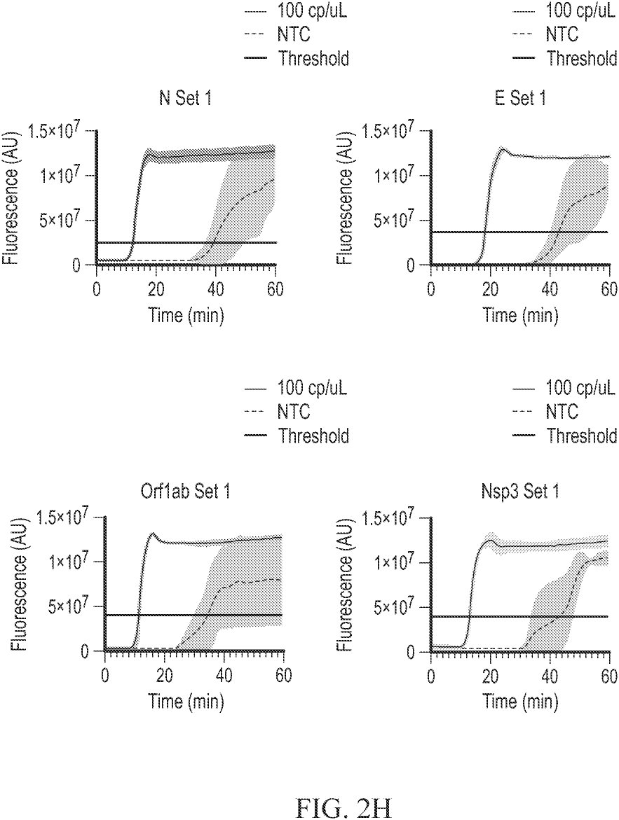

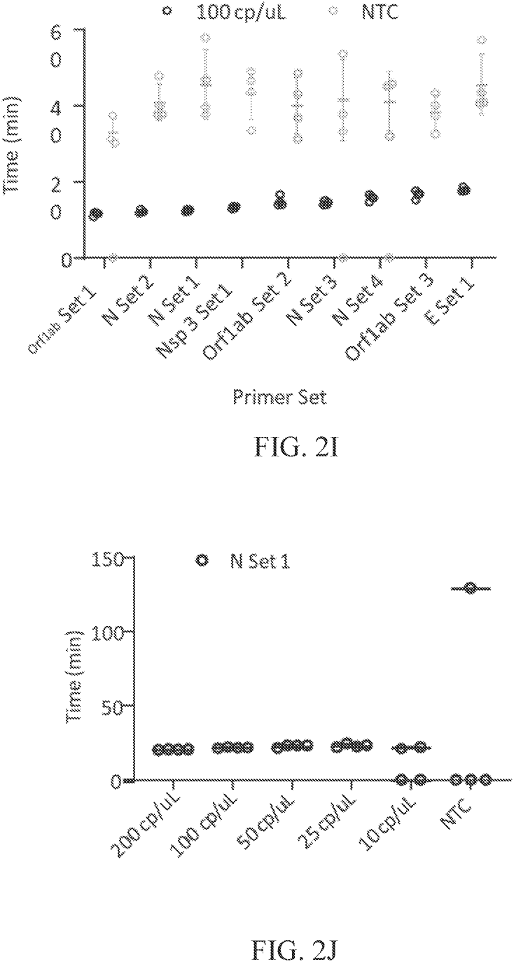

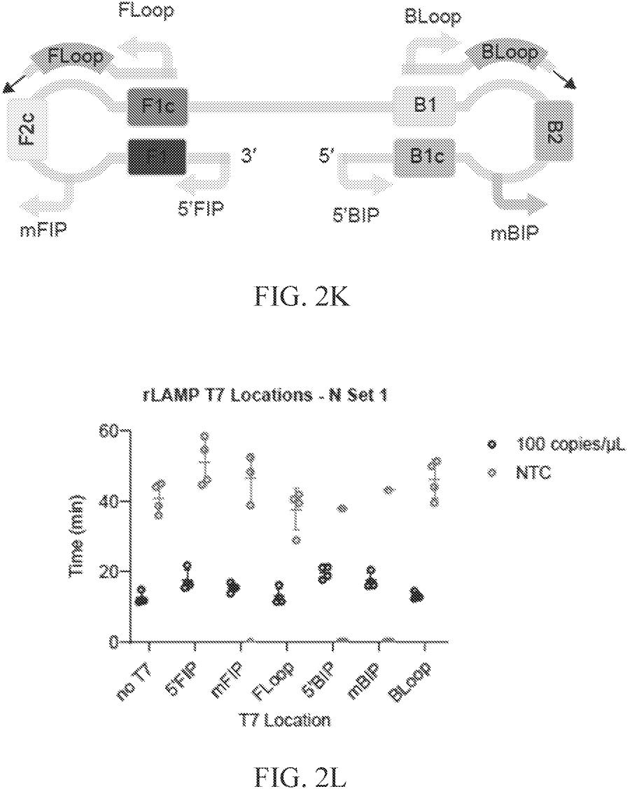

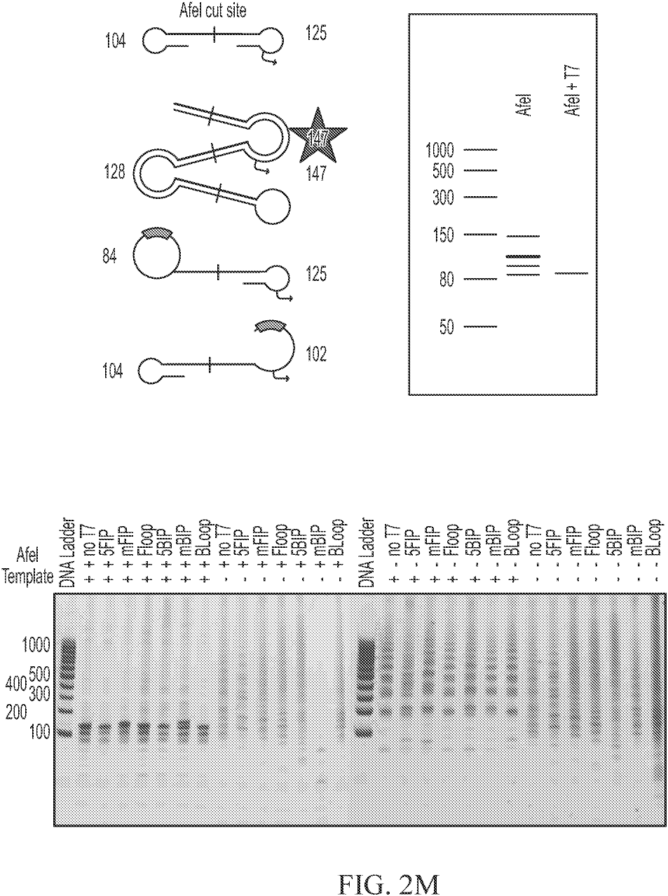

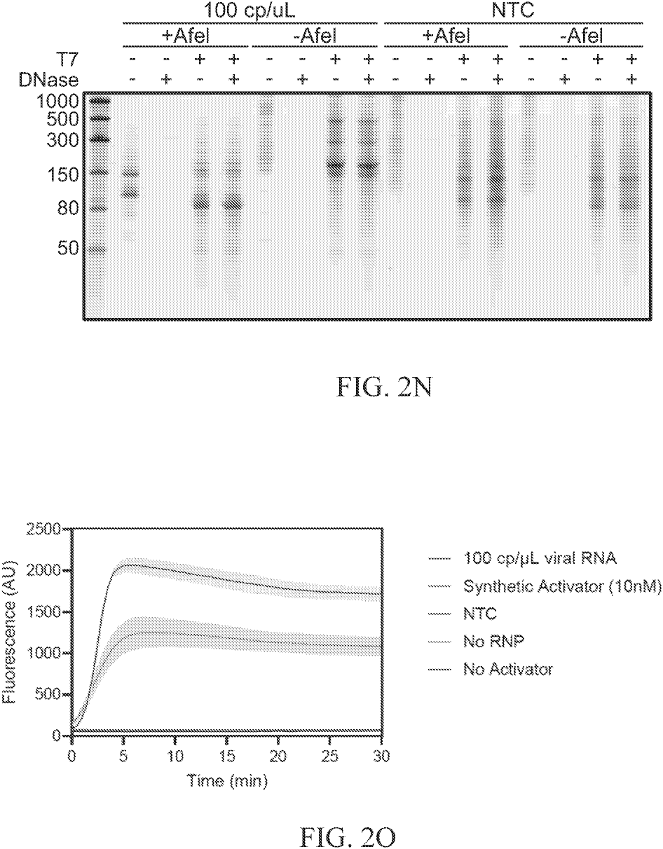

To achieve attomolar sensitivity within 30-40 minutes, we chose LAMP as a cost-effective and rapid method for isothermal amplification. LAMP employs a reverse transcriptase, a strand displacing DNA polymerase, and three primer pairs to convert viral RNA to DNA substrates for LAMP. We screened nine LAMP primer sets targeting distinct regions across the length of the SARS-CoV-2 genome (FIG. 2G, Table 2 below). When targeted to SARS-CoV-2 genomic RNA fragments at 100 copies/uL, all sets resulted in positive LAMP signals. Maximum fluorescence was reached within 20 minutes (FIG. 2H) for all primer sets, and time-tothreshold was determined via the single threshold Cq determination mode as indicated. LAMP primer sets targeting Orflab Set 1, N Set 1, and N Set 2 consistently amplified in under 15 minutes (FIG. 2I).

Each LAMP primer set resulted in a no-template control (NTC) signal, albeit with a delay relative to the positive condition containing viral RNA (FIG. 2H). This high false-positive rate, potentially due to primer dimer formation, can in principle be reduced with a second probe that selectively recognizes the amplified nucleic acid sequence. We therefore sought to combine the sensitivity of LAMP detection with the specificity of Cas13 target recognition.

To avoid Cas13 detection of non-specific amplification, its guide RNAs must have minimal to no sequence overlap with the primer sequences. Due to the complexity of LAMP concatemerization, LAMP primers are highly overlapping and are designed with short amplicons to increase the reaction speed (Notomi (2000) Nucl Acid Res 28(12): E63 doi: 10.1093/nar/28.12.e63). Our LAMP primer sets generated amplicons ranging from 1-60 nt, and so N Set 1, targeting the SARS-CoV-2 N gene, was chosen for further use due to its low time-to-threshold and an amplicon size capable of accommodating Cas13 guide RNAs (FIG. 2I. Table 2). We next performed a dilution series of genomic viral RNA and determined the N Set 1 LOD to be 25 copies/uL (FIG. 2J), comparable with previous studies that report LODs between 10-100 copies/uL (Dao Thi et al. (2020) Science Transl Med 12 (556) doi.org/10.1126/scitranslmed.abc7075; El-Tholoth, Bau (2020) ChemRxiv. ncbi.nlm.nih.gov/pmc/articles/pmc7251958/; Rabe and Cepko (2020) Proc Natl Acad Sci USA 117(39):24450-58).

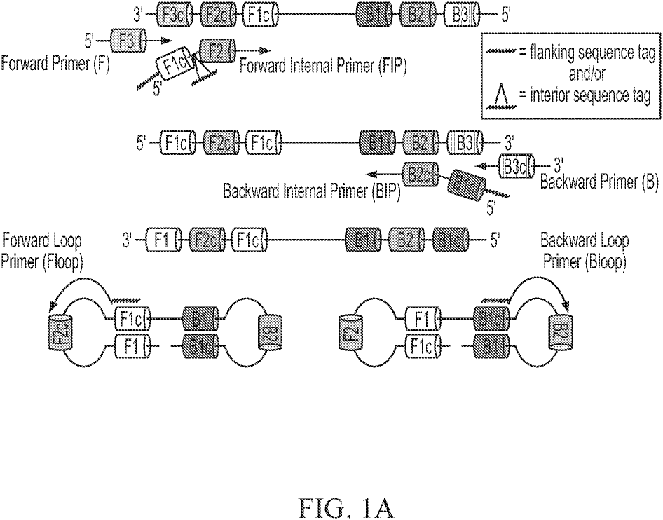

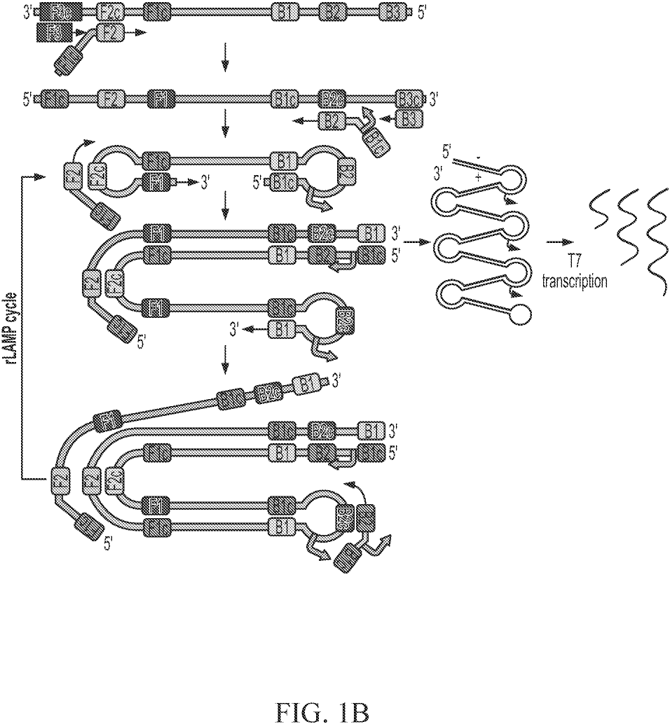

Because Cas13 targets single-stranded RNA (FIG. 2F), while LAMP amplifies DNA substrates, we reasoned that transcription of the LAMP products would enable substrate compatibility. T7 RNA polymerase promoter sequences were incorporated into the LAMP primer sequences to enable subsequent transcription and Cas13 detection. We termed this LAMP amplification to RNA (rLAMP). LAMP employs three primer pairs: forward and backward outer primers (F3/B3) for initial target strand displacement, forward and backward inner primers (FIP/BIP) to form the core LAMP stem-loop structure, and forward and reverse loop primers (Floop/Bloop) for an additional layer of loop-based amplification. Through multiple iterations of primer binding and extension, these stem-loop structures amplify into concatemers composed of inverted repeats of the target sequence (FIG. 1B).

To enable rLAMP, we systematically tested the insertion of T7 promoter sequences in three different regions of LAMP primers - on the 5′ end of the FIP and BIP primers (5′FIP/5′BIP), in the middle of the FIP and BIP primers (mFIP/mBIP), and on the 5′ end of the loop primers (FLoop/BLoop) (FIG. 2K). Addition of the T7 promoter did not greatly affect rLAMP time-tothreshold of N Set 1, given viral genomic RNA at 100 copies/uL (FIG. 2L). To confirm the target sequences were specifically amplified, we performed restriction enzyme digestion on the LAMP products using AfeI, which digests in the Cas13 guide RNA target region within the rLAMP amplicon (FIG. 2M). Lack of AfeI digestion in all NTC conditions confirmed that NTC signal is non-specific amplification lacking the guide-matching target sequence.

To test whether the T7 promoter was properly incorporated and functional in the rLAMP amplicon, we next performed in vitro transcription with T7 RNA polymerase. Denaturing PAGE analysis indicated that the virus template and NTC conditions resulted in significant RNA transcription for all primer sets (FIG. 2N). AfeI digestion of the mBIP rLAMP product should produce a single 147 nt product containing the T7 promoter (FIG. 2M). Subsequent T7 transcription resulted in the expected 85 nt RNA product (FIG. 2N).

In another experimental set, in brief, a target RNA was transcribed into DNA using reverse transcription which was then amplified using LAMP. At the same time, the amount of target was amplified using T7 RNA polymerase where the T7 promoter was incorporated into the primers used for the LAMP DNA amplification. The LAMP amplified product is referred to as the LAMPlicon.

Target RNA samples were analyzed in WarmStart® Colorimetric LAMP 2X Master Mix (New England Biolabs) where the samples were made up at a concentration of 100 copies of target/µL. In addition, T7 polymerase was added at a concentration of 100 ug/uL. The reactions were carried out at 65 degrees, and time to signal threshold was recorded. The primers used are shown below in Table 2 where the T7 promoter sequences are indicated in Bold italic.

Primers were made against the SARS-CoV-2 N gene and the SARS-CoV-2 Nsp3 2-24 gene. Complimentary primer sequences were chosen from the literature (Set 1 N gene: Broughton et al. (2020) Nat Biotechnol. (38)870-874: Set 2 N gene: Joung et al. (2020) doi: 10.1101/2020.05.04.20091231; Set 3 Nsp3 gene: Park et al. (2020) J. Mol Diagn 22(6):729-735) and then the T7 promoter sequences were inserted at either the 5′ end of the primer sequence or inserted into the middle. The location of the primers is indicated where the numbers correspond to the SARS-CoV-2 29867 nucleotide sequence (Park et al. (2020) ibid).

TABLE 2

Primers used for T7 and LAMP amplification

| Gene set | Name | Sequence | Location | SEQ ID NO |
|---|---|---|---|---|
| 1- N gene | T7 5′FIP | GAAATTAATACGACTCACTATAG CGCATTGGCATGGAAGTCACTTTGATGCACCTGTGTAG | 29265..29283 | 6 |
| 1-N | T7 mFIP | CGCATTGGCATGGAAGTCACTAATACGACTCACTATAG TTTGATGGCACCTGTGTAG | 29265..29283 | 7 |
| 1-N | T7 Floop | GAAATTAATACGACTCACTATAG TTCCTTGTCTGATTAGTTC | 29137..29157 | 8 |
| 1-N | T7 5′BIP | GAAATTAATACGACTCACTATAG TGCGGCCAATGTTTGTAATCAGC CAAGGAAATTTTGGGGAC | 29115..29133 | 9 |
| 1-N | T7 mBIP | TGCGGCCAATGTTTGTAATCAGTAATACGACTCACTATAG CCAAG GAAATTTTGGGGAC | 29115..29133 | 10 |
| 1-N | T7 Bloop | GAAATTAATACGACTCACTATAG ACCTTCGGGAACGTGGTT | 29244..29261 | 11 |
| 1-N | FIP | CGCATTGGCATGGAAGTCACTTTGATGGCACCTGTGTAG | | 12 |
| 1-N | BLoop | ACCTTCGGGAACGTGGTT | | 13 |
| 1-N | Floop | TTCCTTGTCTGATTAGTTC | | 14 |
| 2-N | T7 5′FIP | GAAATTAATACGACTCACTATAG GCGGCCAATGTTTGTAATCAGT AGACGTGGTCCAGAACAA | 29095..29112 | 15 |
| 2-N | T7 mFIP | GCGGCCAATGTTTGTAATCAGTTAATACGACTCACTATAG AGACG TGGTCCAGAACAA | 29095..29112 | 16 |
| 2-N | T7 Floop | GAAATTAATACGACTCACTATAG CCTTGTCTGATTAGTTCCTGGT | 29132..29153 | 17 |
| 2-N | T7mBIP #1 | TCAGCGTTCTTCGGAATGTCGTTAATACGACTCACTATAG CTGTGT AGGTCAACCACG | 29255..29272 | 18 |
| 2-N | T7 mBIP #2 | TGCGGCCAATGTTTGTAATCAGTAATACGACTCACTATAG CCAAG GAAATTTTGGGGAC | | 19 |
| 2-N | T7 BIP #1 | GAAATTAATACGACTCACTATAG TCAGCGTTCTTCGGAATGTCGCT GTGTAGGTCAACCACG | 29255..29272 | 20 |
| 2-N | T7 BIP #2 | GAAATTAATACGACTCACTATAG TGCGGCCAATGTTTGTAATCAGC CAAGGAAATTTTGGGGAC | | 21 |
| 2-N | T7 Bloop | GAAATTAATACGACTCACTATAG TGGCATGGAAGTCACACC | 29229..29246 | 22 |
| 2-N | F3 | GCTGCTGAGGCTTCTAAG | | 23 |
| 2-N | FIP | GCGGCCAATGTTTGTAATCAGTAGACGTGGTCCAGAACAA | | 24 |
| 2-N | Bloop | TGGCATGGAAGTCACACC | | 25 |
| 2-N | Floop | CCTTGTCTGATTAGTTCCTGGT | | 26 |
| 3-Nsp3 2-24 | T7 5′FIP | GAAATTAATACGACTCACTATAG TCTGACTTCAGTACATCAAACG AATAAATACCTGGTGTATACGTTGTC | 6276..6298 | 27 |
| 3-Nsp3 2-24 | T7 mFIP | TCTGACTTCAGTACATCAAACGAATTAATACGACTCACTATAG AA ATACCTGGTGTATACGTTGTC | 6276..6298 | 28 |
| 3-Nsp3 2-24 | T7 Floop | GAAATTAATACGACTCACTATAG TGTTTCAACTGGTTTTGTGCTCC A | 6301..6324 | 29 |
| 3-Nsp3 2-24 | T7 5′BIP | GAAATTAATACGACTCACTATAG GACGCGCAGGGAATGGATAATT CCACTACTTCTTCAGAGACT | 6399..6420 | 30 |
| 3-Nsp3 2-24 | T7 mBIP | GACGCGCAGGGAATGGATAATAATACGACTCACTATAG TTCCACT ACTTCTTCAGAGACT | 6399..6420 | 31 |
| 3-Nsp3 2-24 | T7 Bloop | GAAATTAATACGACTCACTATAG TCTTGCCTGCGAAGATCTAAAA C | 6371..6397 | 32 |
| 3-Nsp3 2-24 | F3 | TGCAACTAATAAAGCCACG | | 33 |
| 3-Nsp3 2-24 | FIP | TCTGACTTCAGTACATCAAACGAATAAATACCTGGTGTATACGTT GTC | | 34 |
| 3-Nsp3 2-24 | Bloop | TCTTGCCTGCGAAGATCTAAAAC | | 35 |
| 3-Nsp3 2-24 | Floop | TGTTTCAACTGGTTTTGTGCTCCA | | 36 |
| Orf lab Gene Set 1 | B3 | CTTCTCTGGATTTAACACACTT | | 37 |
| Orf lab Gene Set 1 | BIP | TATTGGTGGAGCTAAACTTAAAGCCCTGTACAATCCCTTTGAGTG | | 38 |
| Orf lab Gene Set 1 | F3 | CGGTGGACAAATTGTCAC | | 39 |
| Orf lab Gene Set 1 | FIP | TCAGCACACAAAGCCAAAAATTTATCTGTGCAAAGGAAATTAAG GAG | | 40 |
| Orf lab Gene Set 1 | Bloop | TTGAATTTAGGTGAAACATTTGTCACG | | 41 |
| Orf lab Gene Set 1 | Floop | TTACAAGCTTAAAGAATGTCTGAACACT | | 42 |
| RNase P Gene Set 1 | RNase P F3 | TTGATGAGCTGGAGCCA | | 43 |
| RNase P Gene Set 1 | RNase P B3 | CACCCTCAATGCAGAGTC | | 44 |
| RNase P Gene Set 1 | RNase P FIP | GTGTGACCCTGAAGACTCGGTTTTAGCCACTGACTCGGATC | | 45 |
| RNase P Gene Set 1 | RNase P BIP | CCTCCGTGATATGGCTCTTCGTTTTTTTCTTACATGGCTCTGGTC | | 46 |
| RNase P Gene Set 1 | RNase P Floop | ATGTGGATGGCTGAGTTGTT | | 47 |
| RNase P Gene Set 1 | RNase P Bloop | CATGCTGAGTACTGGACCTC | | 48 |
| RNase P Gene Set 1 | RNase P FIP M T7 | GTGTGACCCTGAAGACTCGGTAATACGACTCACTATAGTTTTAGC CACTGACTCGGATC | | 49 |
| RNase P Gene Set 1 | RNase P BIP M T7 | CCTCCGTGATATGGCTCTTCGTAATACGACTCACTATAGTTTTTTT CTTACATGGCTCTGGTC | | 50 |
| RNase P Gene Set 1 | RNase FIP 5′T7 | GAAATTAATACGACTCACTATAGGTGTGACCCTGAAGACTCGGTT TTAGCCACTGACTCGGATC | | 51 |
| RNase P Gene Set 1 | RNase BIP 5′ T7 | GAAATTAATACGACTCACTATAGCCTCCGTGATATGGCTCTTCGT TTTTTTCTTACATGGCTCTGGTC | | 52 |
| RNase P Gene Set 1 | RNase Floop 5′ T7 | GAAATTAATACGACTCACTATAGATGTGGATGGCTGAGTTGTT | | 53 |
| RNase P Gene Set 1 | RNase Bloop 5′ T7 | GAAATTAATACGACTCACTATAGCATGCTGAGTACTGGACCTC | | 54 |

The results indicated that the time to threshold concentration of amplified template was not impacted by the presence of the T7 promoter sequences when the template sequence was present. As is typical, in the absence of template sequence (“NTC”) self-primed amplification occurs after a long enough time has elapsed. In FIG. 2. FIG. 2A depicts the results from gene set 1, FIG. 2B shows the results from gene set 2, and FIG. 2C shows the results from gene set 3. Amplification of the template occurred in less than 20 minutes for all primer sets and at all T7 promoter sequence locations.

Analysis of the RNA products following LAMP amplification (FIG. 2D) demonstrated robust transcription and that the banding pattern observed in the no template controls different than the pattern observed in the presence of template.

### Example 2: Detection of the Amplified Target RNA

We first sought to compare the nucleic acid detection properties of Cas13 and Cas 12 enzymes, assessing the reporter cleavage activity of Leptotrichia buccalis Cas 13a (LbuCas13a) and Lachnospiraceae bacterium ND2006 Cas12a (LbCas12a) with matching guide RNA spacer sequences in a dilution series of their corresponding synthetic activator molecules (FIG. 2F, left and middle panels). Cas13 detection was significantly more rapid than Cas12, with a mean of 80-fold and 20-fold faster time to half-maximum fluorescence (FIG. 2F, right panel) at 1 nM and 100 pM activator concentrations, respectively. However, the limit of detection of LbuCas13a is still in the femtomolar range after 60 minutes (East-Seletsky et al. 2017 ibid), which is already beyond the maximum sample-to-answer time that is likely to be relevant for a point-of-care assay.

Next, we optimized buffer conditions to support T7 transcription and Cas13 detection in a single step, followed by systematic screening of Cas13 cleavage activity in the presence of rLAMP products containing T7 promoter insertions in different rLAMP amplicons. We determined that the middle of the BIP primer (mBIP) insertion position resulted in the fastest detection and therefore chose this primer set for further studies (Table 3 below).

TABLE 3

Time to Amplification

| LAMP primer set | Time to Amplification (min) | NTC-Amp time Delta (min) | Amplicon length |
|---|---|---|---|
| Orf1ab Set 1 | 11.6 | 21.3 | 9 |
| N Set 2 | 12.2 | 28.7 | 26 |
| N set 1 | 12.5 | 33.1 | 46 |
| Nsp3 Set 1 | 13.2 | 30.0 | 1 |
| Orflab Set 2 | 14.8 | 25.4 | |

Cas13 rapidly detects the rLAMP amplicon with viral template, while avoiding detection of non-specific NTC amplicons, achieving over 10-fold change in signal over the NTC background in under two minutes (FIG. 2O). Saturating signal was observed within five minutes, reaching over 40-fold signal over background.

To confirm replicability of the DISCoVER pipeline, we tested Cas13 detection on 8 replicates of mBIP rLAMP reactions with activator RNA included at a concentration of 100 copies/uL in a 20 uL reaction. All 8 replicates resulted in over 25 fold-change in signal over NTC, which is stable well beyond the 5 minute detection employed here.

In another set of experiments, to detect the amplified target, Cas13 was utilized in a nuclease chain reaction. In this system, the target RNA sequence is recognized by a guide RNA/Cas13 complex (RNP). The recognition of the target releases trans, non-specific RNase activity by the activated Cas13 complex which then acts on a single stranded RNA reporter molecule. The reporter molecule comprises a fluorescent molecule linked by the single stranded RNA to a quencher molecule such that prior to cleavage, the fluorescence is quenched. Upon cleavage, the fluorescent reporter is released and fluoresces and can be detected.

In these experiments, 2-3 µL of the amplification reaction from Example 1 was added to the Cas13 detection reaction. The reaction was carried out in the Cas13 sensing buffer: 5X Cas 13a Buffer 7 (East-Selesky et al. (2016) Nature 538(7624):270-273), 100 mM HEPES (pH 6.8), 250 mM KCl, 25 mM MgCl2, 500 µg/mL BSA (UltraPure), 0.05% Igepal CA-630, 10% glycerol. LbuCas132 was added at 10-50 nM, and guide RNA was added at a 1:1 or 2:1 ratio of the protein concentration. The fluorescent signal and time were then measured.

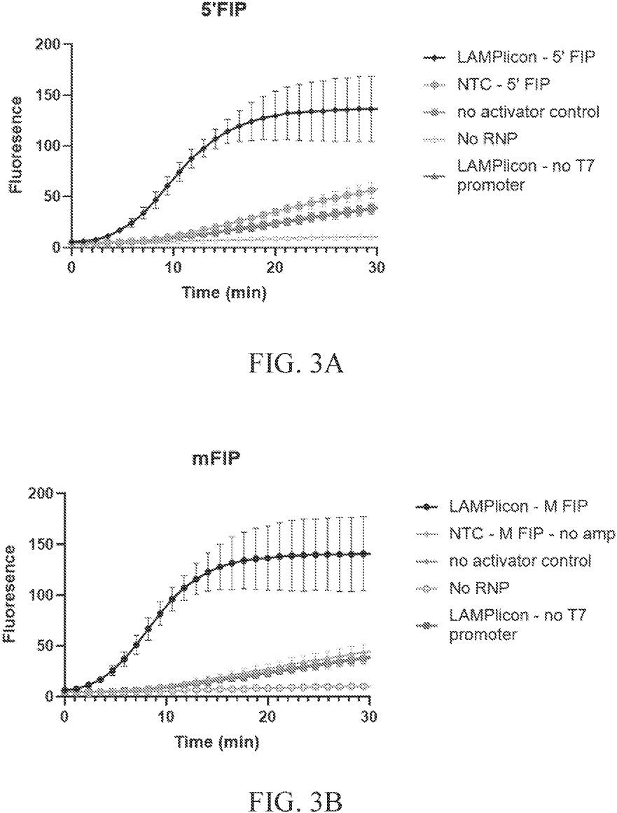

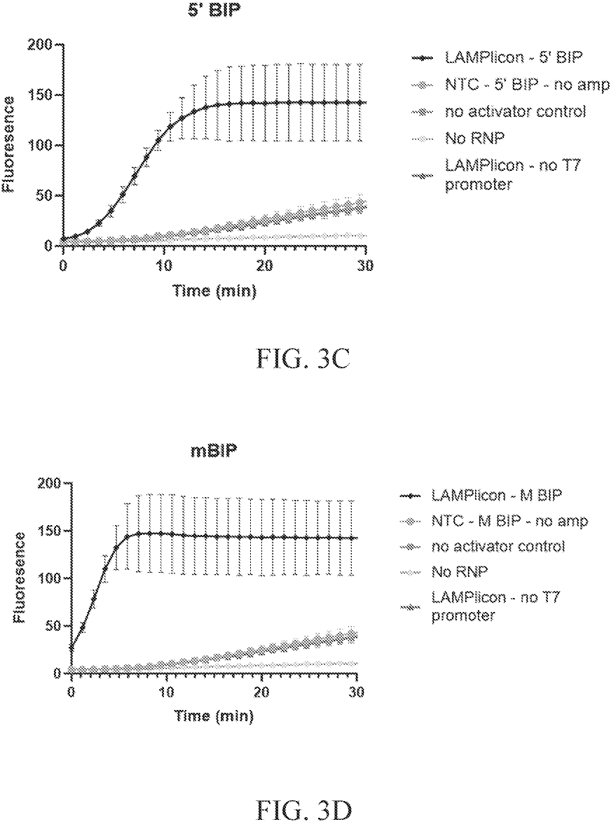

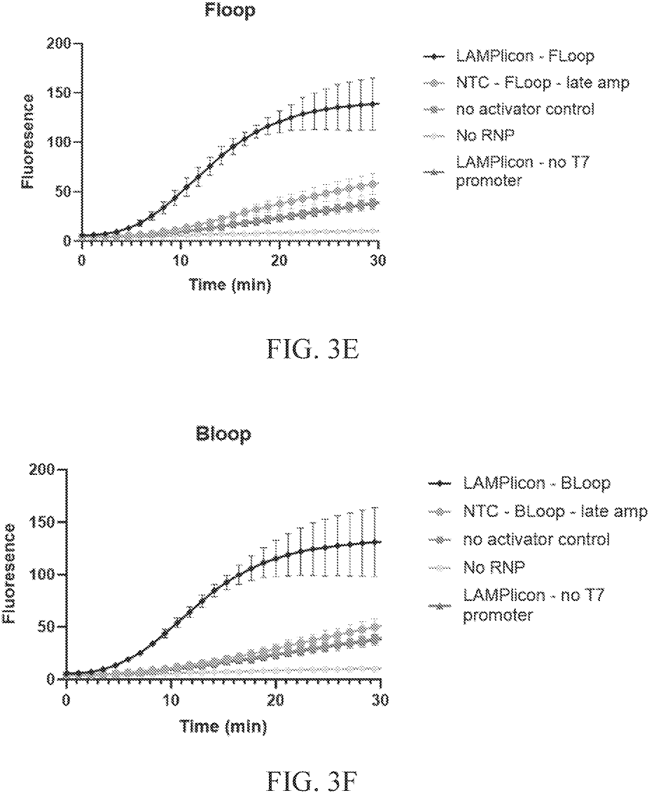

The results (FIG. 3) demonstrated that the when the LAMPlicon is included in the reaction, it is able to act as an activator for the Cas13 RNP, demonstrating that the target nucleic acid was in the tested sample. Shown in each graph are the results using primers where one primer comprises the T7 promoter sequences. For example, FIG. 3A depicts the results observed when the gene set 1 5′FIP primer comprises the T7 promoter sequence. As can be seen, a comparison of the speed and amount of signal detected is much higher when the primer comprises the T7 promoter sequences. Control traces indicate experiments that lack the activator target for the Cas13 RNP (“no activator control”), that lack the Cas13 detector (“no RNP”), that lack the T7 promoter sequences in the primer (“LAMPlicon- no T7 promoter) and that lack the target sequence (“NTC”). The data demonstrates that the Cas13 system was able to detect the SARS-CoV-2 target sequence at 10 copies per µL.

### Example 3: Saliva Detection Protocol

With an amplification and detection protocol in place, we next optimized sample processing to establish a simple protocol of heat paired with chemical reagents to promote viral inactivation and dampen the activity of RNA-degrading nucleases present in saliva (Ostheim et al. (2020) Sci Reports 10(1):11147). We assayed different concentrations of the shelf-stable reducing agent TCEP (Tris (2 carboxyethyl)phosphine) paired with the ion chelator EDTA (ethylenediaminetetraacetic acid), commercially available reagents such as QuickExtract buffers containing detergents and proteinase, and DNA/RNA shield containing chaotropic guanidine thiocyanate.

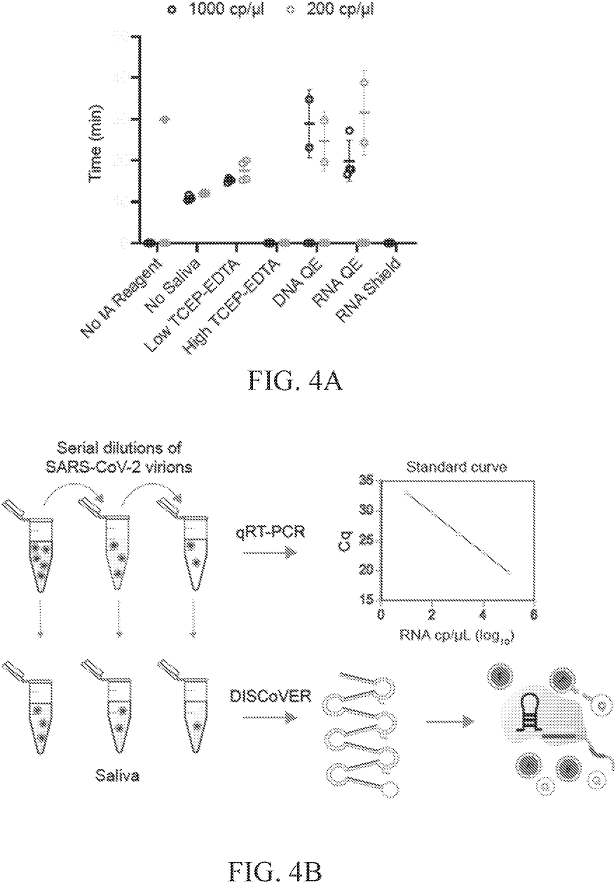

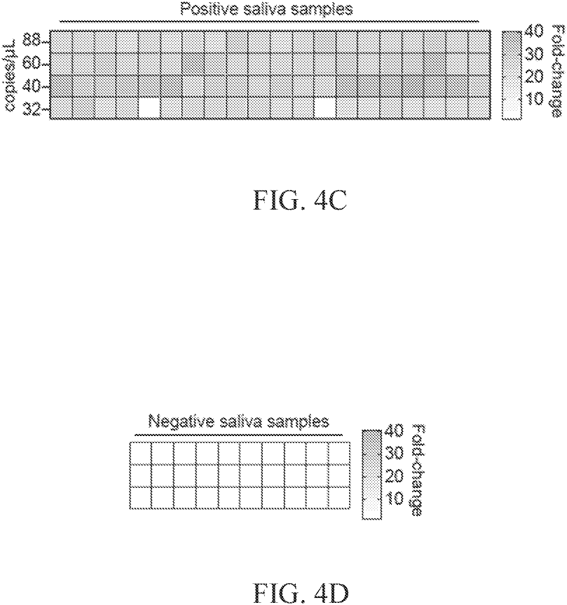

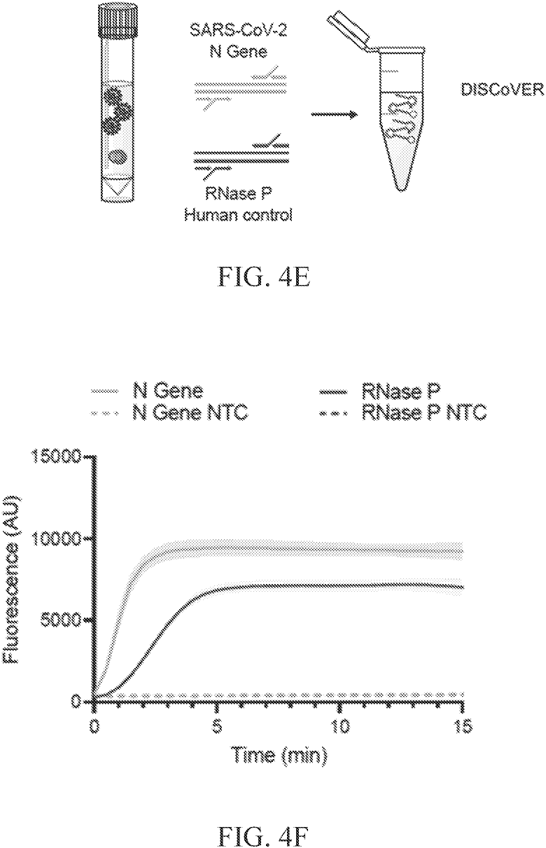

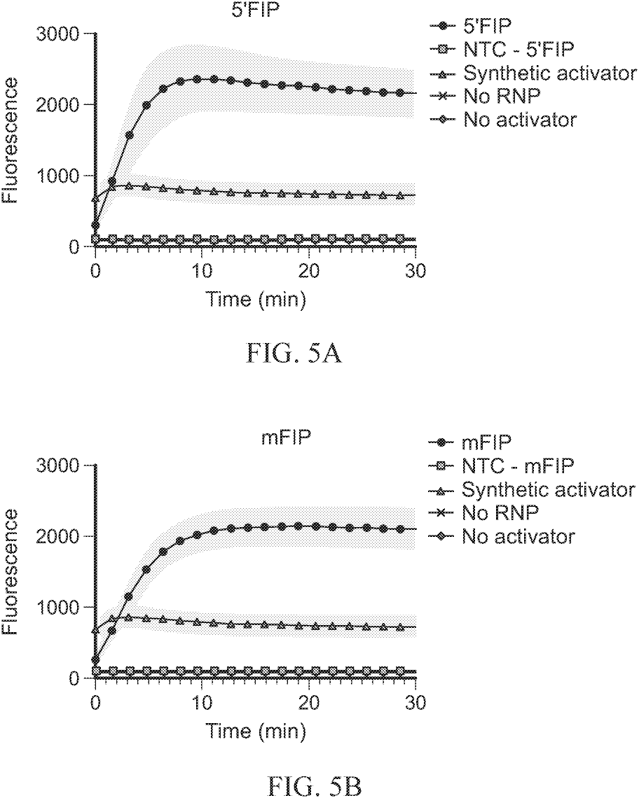

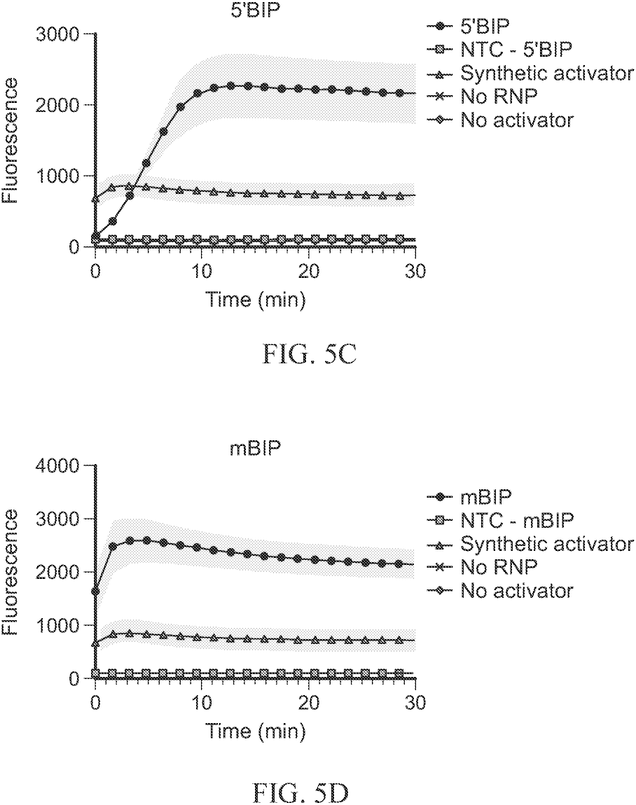

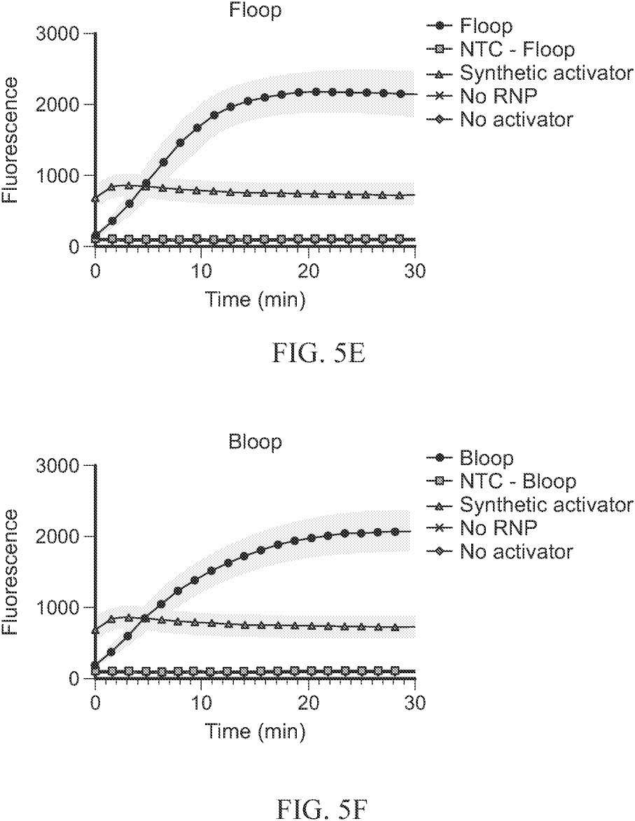

To test the compatibility of these inactivating reagents with rLAMP, we created mock positive saliva samples by adding the reagents to heat-treated saliva (Chin et al. (2020) Lancet. Microbe 1(1):e10) and adding SARS-CoV-2 genomic RNA at two different concentrations: 1000 cp/uL and 200 cp/uL. In the absence of inactivating reagents, we were unable to detect any rLAMP signal, suggesting degradation of RNA in saliva by endogenous RNases present in the sample. In contrast, genomic RNA without saliva was rapidly amplified in under 15 min. Only the low concentration condition of TCEP-EDTA was capable of protecting target RNA and preserving rLAMP sensitivity in all 4 replicates (FIG. 4A). This reagent cocktail simultaneously breaks protein disulfide bonds and sequesters divalent cations. Its activity is expected to dampen RNase activity while simultaneously disrupting mucin gel formation, reducing variable saliva viscosity for simpler sample processing (Meldrum et al. (2018) Sci Reports 8(1):5802; Tabachnik et al. (1981) J Biol Chem 256 (14):7161-65).

Finally, we used the guidelines provided by the FDA for Emergency Use Authorization (EUA) in May 2020 to determine the analytical sensitivity and specificity for DISCoVER on saliva samples (Food and Drug Administration, 2020). To determine the limit of detection, viral stocks were serially diluted in media and quantified with RT-qPCR relative to a standard curve generated from synthetic genomic RNA. These known concentrations of virus were spiked into negative saliva samples collected before November 2019 in BSL3 conditions and run through the DISCoVER workflow (FIG. 4B). We performed 20 DISCoVER replicates for a range of virus concentrations, determining 40 cp/uL of virus in directly lysed saliva (FIG. 4C) to be the lowest concentration tested where at least 19/20 replicates amplified successfully. To assay DISCoVER specificity, saliva samples from 30 different individuals negative for SARS-CoV-2 were tested without any false positive signal (FIG. 4D).

In point-of-care settings, successful sample inactivation and lysis would ideally be confirmed by an internal process control (FIG. 4E). This is particularly important for successful saliva-based population screening, as some individuals can have high viscosity or mucin gel loads in their samples. To achieve this, we multiplexed detection of the SARS-CoV-2 N gene and the human RNase P gene in the rLAMP amplification step. Cas13 detection of RNase P on saliva samples also reached saturation within 5 minutes, enabling confirmation of saliva RNase inactivation and viral lysis (FIG. 4F).

Here, we have demonstrated an RNA extraction-free workflow for the facile detection of SARSCoV-2 virus. DISCoVER’s combination of a 20-30 minute LAMP step followed by T7 transcription and Cas13-based detection creates a rapid testing protocol with attomolar sensitivity and high specificity. As each LAMP product can serve as a substrate for transcription initiation, peak Cas13 signal occurs in under five minutes due to rapid generation of nanomolar substrate concentrations. The combination of sensitive nucleic acid amplification with CRISPR-mediated specificity and programmability enables flexible diagnostics for diverse pathogen detection.

We demonstrate the DISCoVER system on unextracted saliva, with the aim of enabling the first key steps for a point-of care diagnostic. A saliva-based assay may not require medical personnel for sample collection, as is preferable for nasopharyrogeal swabs, and the increased comfort of sample collection will likely incentivize patient compliance and commitment to frequent testing. It has also been shown that saliva is a reliable sample matrix for asymptomatic testing in community surveillance, as saliva samples have comparable viral titers to NP swabs (Wyllie et al. 2020, ibid). To diversify sampling supply chain. DISCoVER can also be applied to other samples such as nasopharyngeal swabs and self-administered anterior nares swabs.

In comparison with other CRISPR detection methods such as DETECTR and STOPCovid V2, DISCoVER does not employ a sample extraction or purification step (Broughton et al. (2020) Nat Biotech 38 (7): 870-74; Joung et al. (2020) New Engl J Med 383(15): 1492-94), eliminating reliance on commercial RNA extraction kits. The direct lysis method employed here exploits common reagents for chemical reduction and ion chelation that are simple, widely available at low cost, and stable at room temperature. DISCoVER maintains attomolar sensitivity comparable to other CRISPR detection-based assays that include viral RNA extraction and purification (Broughton et al. (2020) ibid; Patchsung et al. (2020) Nat Biomed Eng 4(12):1140-1149; Fozouni et al. 2020 MedRxiv.org/10.1101/2020.09.28.20201947).

Finally, we demonstrate multiplexed SARS-CoV-2 detection with a human internal control during the DISCoVER amplification stage. This can be adapted for multi-color detection using Cas enzymes with orthogonal cleavage motifs, acting on reporters for distinct fluorescent channels (Gootenberg et al. (2018) Science 360 (6387):439-44). Higher-order multiplexing can also be used for the detection of influenza types A and B and other common respiratory viruses that would be desirable to detect in a single test. Because the core DISCoVER enzymes are able to convert between any RNA and DNA sequence, any pathogen that is inactivated and lysed by our protocol can be detected. Saliva sample collection, lack of viral RNA extraction, and the capability for target multiplexing lends DISCoVER favorable properties for a point-of-care diagnostic.

### Example 4: Exemplary Multiplex rLAMP Assay

To demonstrate multiplex detection, the following experiment was performed using regents shown in Tables 4A and 4B. Primers were synthesized to amplify either the human RNAse P or the SARS-CoV-2 N gene.

### Multiplexed rLAMP

TABLE 4A

Conditions opted to the final concentrations below in a 42 µL reaction

| | |
|---|---|
| Component | Final Concentration |
| 2x NEB WarmStart LAMP MasterMix | 1x |
| 10x Set 1 FIP/BIP Primers | 1.6 μΜ |
| 10x Set 1 F3/B3 Primers | 0.2 µM |
| 10x Set 1 Floop/Bloop Primers | 0.4 μΜ |
| 10x Set 2 FIP/BIP Primers | 0.8 µM |
| 10x Set 2 F3/B3 Primers | 0.1 µM |
| 10x Set 2 Floop/Bloop Primers | 0.4 μΜ |

The remaining volume consisted of the rLAMP target (either in water or saliva).

### Multiplexed Cas13 Detection

TABLE 4B

Conditions opted to the final concentrations below

| | |
|---|---|
| Component | Final Concentration |
| LbuCas13a | 1 nM |
| LbaCas13a | 10 nM |
| penta-U FAM reporter | 200 nM |
| penta-A HEX reporter | 200 nM |
| T7 | 0.1 mg/mL |
| MgCl2 | 25 mM |
| DTT | 10 mM |
| NTPs (25 mM) | 5 mM |
| 5x Buffer | 1x |

2 µL of N Gene LAMPlicon and RNase P LAMPlicon each were added to bring the total reaction volume to 20 µL.

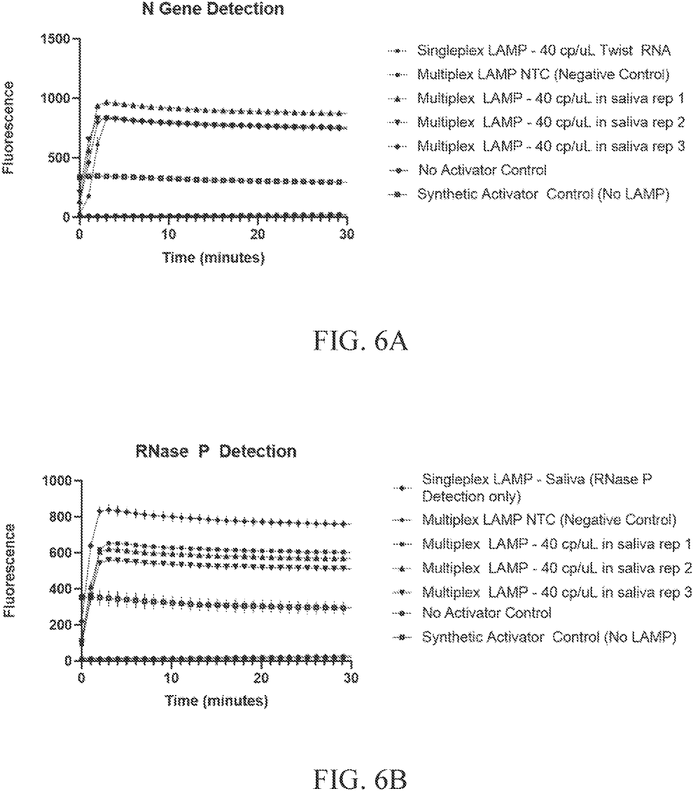

The experiment demonstrated (FIG. 6A) that Cas13 complexed with the N Gene LAMPlicon targeting guide was able to detect the amplified material at the previously established limit of detection (LOD) of 40 cp/uL. Similarly, FIG. 6B shows detection of the RNase P LAMPlicon with Cas13 complexed with RNase P targeting guide. This experiment shows that multiplexed LAMP amplification to simultaneously produce detectable LAMPlicon amplification products was achieved in a single reaction.

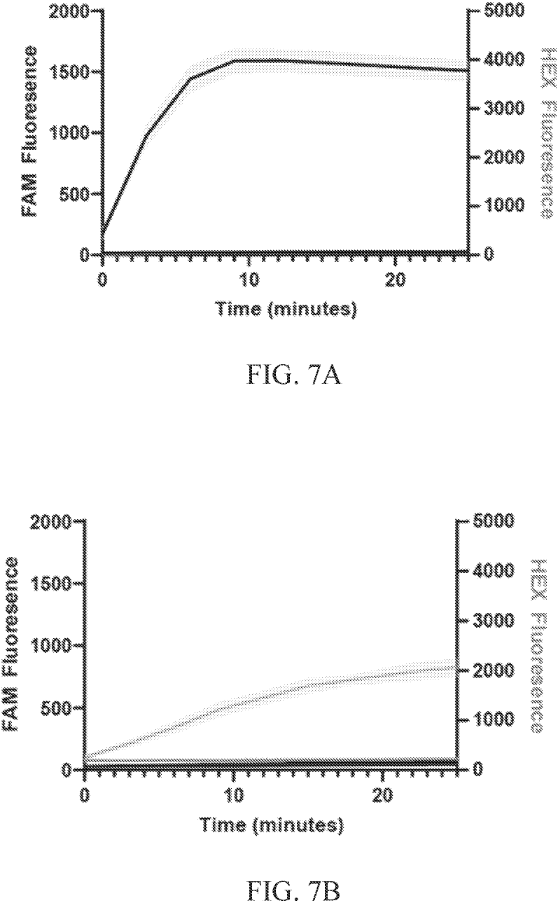

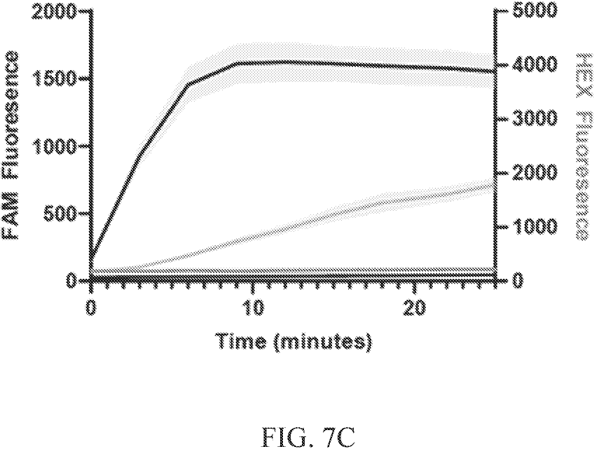

Next, we sought to demonstrate that our system could detect the two LAMPlicons that had been produced above. To achieve this, we used two different reporter molecules (5-U FAM (IDT) and 5-A HEX (IDT)) as shown in Table 4B above. An experiment was performed that included the N Gene and RNase P LAMPlicons, the FAM and HEX reporters, and 1 nM LbuCas13a complexed with N Gene targeting guide. The results (FIG. 7A) showed that only expression of the FAM reporter signal was seen, indicating that LbuCas 13a specifically and robustly detected only the N Gene LAMPlicon without any off-target detection of the RNase P LAMPlicon that would have resulted in background HEX reporter cleavage. Similarly, we were able to detect the RNase P LAMPlicon in an experiment that included the N Gene and RNase P LAMPlicons, FAM and HEX reporters, and 10 nM LbaCas13a complexed with RNase P targeting guide (FIG. 7B). Finally, we were able to perfonn simultaneous detection of the N-gene LAMPlicon and RNase P LAMPlicons by combining the materials in the two separate experiments above into a single reaction (FIG. 7C).

TABLE 5

Primer and Guide RNA Sequences

| Primer Name | Sequence | SEQ ID NO |
|---|---|---|
| ACTB Set 1 F3 | AAGATGAGATTGGCATGGC | 55 |
| ACTB Set 1 B3 | GCAAGGGACTTCCTGTAAC | 56 |
| ACTB Set 1 FIP | CTCCAACCGACTGCTGTCTTTGGCTTGACTCAGGATTT | 57 |
| ACTB Set 1 BIP | CCCAAAGTTCACAATGTGGCCGCATCTCATATTTGGAATGAC | 58 |
| ACTB Set 1 LoopF | ACCTTCACCGTTCCAGTT | 59 |
| ACTB Set 1 LoopB | GGACTTTGATTGCACATTGTTG | 60 |
| ACTB Set 1 T7 M FIP | CTCCAACCGACTGCTGTCTAATACGACTCACTATAGTTTGGCTTGACT CAGGATTT | 61 |
| ACTB Set 1 T7 M BIP | CCCAAAGTTCACAATGTGGCCGTAATACGACTCACTATAGCATCTCAT ATTTGGAATGAC | 62 |
| ACTB Set 1 T7 5 FIP | GAATTAATACGACTCACTATAGCTCCAACCGACTGCTGTCTTTGGCTT GACTCAGGATTT | 63 |
| ACTB Set 1 T7 5 BIP | GAAATTAATACGACTCACTATAGCCCAAAGTTCACAATGTGGCCGCAT CTCATATTTGGAATGAC | 64 |
| ACTB Set 1 T7 5 FLoop | GAAATTAATACGACTCACTATAGACCTTCACCGTTCCAGTT | 65 |
| ACTB Set 1 T7 5 BLoop | GAAATTAATACGACTCACTATAGGGACTTTGATTGCACATTGTTG | 66 |
| GAPDH Set 5 F3 | GTGAAGGTCGGAGTCAAC | 67 |
| GAPDH Set 5 B3 | GACTCCACGACGTACTCA | 68 |
| GAPDH Set 5 FIP | TGGGTGGAATCATATTGGAACAGGATATTGTTGCCATCAATGAC | 69 |
| GAPDH Set 5 BIP | ACCGTCAAGGCTGAGAACGATCTCGCTCCTGGAAGA | 70 |
| GAPDH Set 5 LoopF | AACCATGTAGTTGAGGTCAATG | 71 |
| GAPDH Set 5 LoopB | GGAAGCTTGTCATCAATGGAA | 72 |
| GAPDH Set 5 T7 M FIP | TGGGTGGAATCATATTGGAACATAATACGACTCACTATAGGGATATTG TTGCCATCAATGAC | 73 |
| GAPDH Set 5 T7 M BIP | ACCGTCAAGGCTGAGAACGTAATACGACTCACTATAGATCTCGCTCCT GGAAGA | 74 |
| GAPDH Set 5 T7 5 FIP | GAAATTAATACGACTCACTATAGTGGGTGGAATCATATTGGAACAGGA TATTGTTGCCATCAATGAC | 75 |
| GAPDH Set 5 T7 5 BIP | GAAATTAATACGACTCACTATAGACCGTCAAGGCTGAGAACGATCTCG CTCCTGGAAGA | 76 |
| GAPDH Set 5 T7 5 FLoop | GAAATTAATACGACTCACTATAGAACCATGTAGTTGAGGTCAATG | 77 |
| GAPDH Set 5 T7 5 BLoop | GAAATTAATACGACTCACTATAGGGAAGCTTGTCATCAATGGAA | 78 |
| GAPDH Set 9 F3 | GCGCTGCCAAGGCTGT | 79 |
| GAPDH Set 9 B3 | CCCAGGATGCCCTTGAGG | 80 |
| GAPDH Set 9 FIP | GTGGGGACACGGAAGGCCAGGGCAAGGTCATCCCTGA | 81 |
| GAPDH Set 9 BIP | TGCCAACGTGTCAGTGGTGGACTGCTTCACCACCTTCTTGA | 82 |
| GAPDH Set 9 LoopF | CCAGTGAGCTTCCCGTTCAGC | 83 |
| GAPDH Set 9 LoopB | TGCCGTCTAGAAAAACCTGCCA | 84 |
| GAPDH Set 9 T7 M FIP | GTGGGGACACGGAAGGCCATAATACGACTCACTATAGGGGLAAGGTCA TCCCTGA | 85 |
| GAPDH Set 9 T7 M BIP | TGCCAACGTGTCAGTGGTGGACTAATACGACTCACTATAGTGCTTCAC CACCTTCTTGA | 86 |
| GAPDH Set 9 T7 5 FIP | GAAATTAATACGACTCACTATAGGTGGGGACACGGAAGGCCAGGGCAA GGTCATCCCTGA | 87 |
| GAPDH Set 9 T7 5 BIP | GAAATTAATACGACTCACTATAGTGCCAACGTGTCAGTGGTGGACTGC TTCACCACCTTCTTGA | 88 |
| GAPDH Set 9 T7 5 FLoop | GAAATTAATACGACTCACTATAGCCAGTGAGCTTCCCGTTCAGC | 89 |
| GAPDH Set 9 T7 5 BLoop | GAAATTAATACGACTCACTATAGTGCCGTCTAGAAAAACCTGCCA | 90 |
| RPP30 FIP | agtttctccatggagaagcgcttttctgtttgggctctctgaa | 91 |
| RPP30 BIP | tatctctacagtgaagaaacctcggttttttcttggaagctggaagac | 92 |
| RPP30 F3 | tgacgtggcaaatctagg | 93 |
| RPP30 B3 | actggggcattcttttcag | 94 |
| Rpp30 LF | gttggtggacaccgc | 95 |
| RPP30 LB | ccatcagaaggagatgaagatt | 96 |
| SC_RPP30 T7 M FIP | agtttctcccatggagaagcgctTAATACGACTCACTATAGtttctgtt tgggctctctgaa | 97 |
| SC_RPP30 T7 M BIP | tatctctacagtgaagaaacctcggTAATACGACTCACTATAGttttt tcttggaagctggaagac | 98 |
| SC_RPP30 T7 5 FIP | GAAATTAATACGACTCACTATAGagtttctccatggagaagcgctttt ctgtttgggctctctgaa | 99 |
| SC_RPP30 T7 5 BIP | GAAATTAATACGACTCACTATAGtatctctacagtgaagaaacctcgg ttttttcttggaagctggaagac | 100 |
| SC_RPP30 T7 5 FLoop | GAAATTAATACGACTCACTATAGgttggtggacaccgc | 101 |
| SC_RPP30 T7 5 BLoop | GAAATTAATACGACTCACTATAGcccatcagaagaagatgaagatt | 102 |
| 18 s rRNA FIP | tggcctcagttccgaaaaccaattttcctggataccgcagctagg | 103 |
| 18 s rRNA BIP | ggcattcgtattgcgccgctttttggcaaatgctttcgctctg | 104 |
| 18 s rRNA F3 | gttcaaagcaggcccgag | 105 |
| 18 s rRNA B3 | cctccgactttcgttcttga | 106 |
| 18 s rRNA LF | agaaccgcggtcctattccattatt | 107 |
| 18 s rRNA LB | attcttggaccggcgcaag | 108 |
| 18 s tRNA T7 M FIP | tggcctcagttccgaaaaccaatTAATACGACTCACTATAGtttcctg gataccgcagctagg | 109 |
| 18 s rRNA T7 M BIP | ggcattcgtattgcgccgcTAATACGACTCACTATAGtttttggcaaa tgctttcgctctg | 110 |
| 18 s rRNA T7 5 FIP | GAAATTAATACGACTCACTATAGtggcctcagttccgaaaaccaattt tcctggataccgcagctagg | 111 |
| 18 s rRNA T7 5 BIP | GAAATTAATACGACTCACTATAGggcattctgtattgctgccgctttttg gcaaatgctttcgctctg | 112 |
| 18 s rRNA T7 5 FLoop | GAAATTAATACGACTCACTATAGagaaccgcggttcctattccattatt | 113 |
| 18 s rRNA T7 5 BLoop | GAAATTAATACGACTCACTATAGattcttggaccggcgcaag | 114 |
| ACTB activator | GTCGGTTGGAGCGAGCATCCCCCAAAGTTCAC | 115 |
| ACTB guide 1 | UAGACCACCCCAAAAAUGAAGGGGACUAAAACGTGAACTTTGGGGGAT GCTCG | 116 |
| ACTB guide 2 | UAGACCACCCCAAAAAUGAAGGGGACUAAAACGAACTTTGGGGGATGC TCGCT | 117 |
| ACTB guide 3 | UAGACCACCCCAAAAAUGAAGGGGACUAAAACACTTTGGGGGATGCTC GCTCC | 118 |
| GAPDH9 activator | TGGCCTTCCGTGTCCCCACTGCCAACGTGTCAGTGG | 119 |
| GAPDH9 guide 1 | UAGACCACCCCAAAAAUGAAGGGGACUAAAACGTTGGCAGTGGGGACA CGGA | 120 |
| GAPDH9 guide 2 | UAGACCACCCCAAAAAUGAAGGGGACUAAAACCACGTTGGCAGTGGGG ACAC | 121 |
| GAPDH9 guide 3 | UAGACCACCCCAAAAAUGAAGGGGACUAAAACTGACACGTTGGCAGTG GGGA | 122 |
| GAPDH5 activator | TTCCACCCATGGCAAATTCCATGGCACCGTCAAG | 123 |
| GAPDH5 guide 1 | UAGACCACCCCAAAAAUGAAGGGGACUAAAACACGGTGCCATGGAATT TGCCA | 124 |
| GAPDH5 guide 2 | UAGACCACCCCRAAAAAUGAAGGGGACUAAAACGGTGCCATGGAATTTG CCATG | 125 |
| GAPDH5 guide 3 | UAGACCACCCCAAAAAUGAAGGGGACUAAAACTGCCATGGAATTTGCC ATGGG | 126 |
| LbaCas13a Rnase P guide | AGAAGAUAGCCCAAGAAAGAGGGCAAUAACACAUUCCGAAGAACGCUG AAGCGC | 127 |

Note: “ACTB” is the human beta-actin gene, “GAPDH” is the human glyceraldehyde 3-phosphate dehydrogenase gene. “RPP” is the human RNase P.

What is claimed is:

1. A system for amplifying at least one target nucleic acid sequence, the system comprising:
(a) at least one target DNA sequence obtained from a sample, optionally wherein the target DNA sequence is transcribed from RNA obtained from the sample;
(b) at least four primers including:
(i) a forward inner primer (FIP) that binds to the target DNA sequence;
(ii) a backward inner primer (BIP) that binds to the target DNA sequence;
(iii) a forward outer primer (F3), wherein the forward outer primer binds to the target DNA sequence 5′ to the FIP;
(iv) a backward outer primer (B3), wherein the backward outer primer binds to the target DNA sequence 3′ to the BIP; optionally further comprising
(v) a forward loop primer (floop); and
(vi) a backward loop primer (bloop);
wherein at least one of the primers comprises a sequence tag, wherein the sequence tag is an RNA polymerase promoter sequence, such that in the presence of a DNA polymerase, the target DNA sequence is amplified to generate amplicons comprising the sequence tag, optionally wherein the amplicons comprise a detectable label.

2. The system of claim 1, further comprising
(c) RNA substrate amplicons transcribed from the amplicons comprising the RNA polymerase promoter sequence.

3. The system of claim 1, wherein the sequence tag is 5′ of the primer.

4. The system of claim 1, wherein the sequence is interior in the primer.

5. The system of claim 1, wherein the sequence tag is included in the FIP, BIP, flop, or bloop primer.

6. The system of claim 1, wherein at least one primer comprises a primer as shown in Table 2 or Table 5.

7. A method of amplifying one or more target sequences in a sample, the method comprising generating amplicons using the system of claim 1.

8. The method of claim 7, further comprising detecting one of more of the amplicons.

9. The method of claim 8, wherein the one of more amplicons are detected using the detectable label.

10. The method of claim 7, further comprising detecting the amplicons using a CRISPR-Cas detection system.

11. The method of claim 7, further comprising multiplex detection of two or more different amplicons.

12. The method of claim 7, wherein the amplicons are generated within 2 hours or less.

13. The system of claim 1, wherein the at least one target nucleic acid sequence is obtained from the group consisting of a mammal, a virus, a bacterium, and a fungus.

14. The system of claim 13, wherein the virus is a coronavirus.

15. The system of claim 1, wherein the sample is a biological or environmental sample .

16. The method of claim 7 further comprising quantifying the levels of the detectable label.

17. A kit comprising the system of claim 1 and instructions for use.

18. The system of claim 14, wherein the coronavirus is a SARS-CoV-2 coronavirus.

19. The system of claim 15, wherein the biological sample is selected from the group consisting of blood, saliva, urine, biopsy, plasma, serum, bronchoalveolar lavage, sputum, a fecal sample, cerebrospinal fluid, a fine needle aspirate, a swab sample, interstitial fluid, synovial fluid, nasal discharge, tears, buffy coat, a mucous membrane sample, and an epithelial cell sample.

20. The system of claim 15, wherein the environmental sample is selected from the group consisting of a sample obtained from a surface, a sample obtained from the air, a sample obtained from a sewage system, and a sample obtained from a waste water treatment facility.
